(19) United States
(12) Patent Application Publication
(10) Pub. No.: US 2026/0029397 A1
(43) Pub. Date: Jan. 29, 2026

(54) A METHOD FOR PREDICTING THE RESPONSE OF METASTASES TO IMMUNOTHERAPY IN A METASTATIC COLORECTAL CANCER PATIENT

(71) Applicant: INSTITUT CLAUDIUS REGAUD, TOULOUSE (FR)

(72) Inventors: Maha AYYOUB, TOULOUSE (FR); Christel DEVAUD, CUGNAUX (FR)

(21) Appl. No.: 18/995,475

(22) PCT Filed: Jul. 20, 2023
(86) PCT No.: PCT/EP2023/070223

§ 371 Date: Jan. 16, 2025

(30) Foreign Application Priority Data
Jul. 21, 2022 (EP) 22306090.6

(51) Int. Cl. G01N 33/50

(52) U.S. Cl. CPC G01N 33/505; G01N 33/5055; G01N 2333/70517; G01N 2333/70553; G01N 2333/70596; G01N 2800/52

(57) ABSTRACT

The present disclosure relates to a method for predicting the response of a metastasis to immunotherapy in a metastatic colorectal cancer patient. The present disclosure also encompasses an immunotherapy for use in the treatment of a metastatic colorectal cancer in a patient in need thereof previously identified as having a metastasis responsive to immunotherapy and an immune-stimulating agent for use in the treatment of a metastatic colorectal cancer in a patient in need thereof previously identified as having a metastasis non-responsive to immunotherapy. The present disclosure also relates to a pharmaceutical composition comprising an immune-stimulating agent and retinoic acid for use in the treatment of a metastatic colorectal cancer in a patient in need thereof.

### FIELD OF THE INVENTION

The present disclosure relates to a method for predicting the response of a metastasis to immunotherapy in a metastatic colorectal cancer patient. The present disclosure also encompasses an immunotherapy for use in the treatment of a metastatic colorectal cancer in a patient in need thereof previously identified as having a metastasis responsive to immunotherapy and an immune-stimulating agent for use in the treatment of a metastatic colorectal cancer in a patient in need thereof previously identified as having a metastasis non-responsive to immunotherapy. The present disclosure also relates to a pharmaceutical composition comprising an immune-stimulating agent and retinoic acid for use in the treatment of a metastatic colorectal cancer in a patient in need thereof.

### BACKGROUND OF THE INVENTION

Primary colorectal tumors are located within the gut and their distant metastases predominantly occur in the liver. Up to 25% of colorectal cancer (CRC) patients are diagnosed at stage IV metastatic disease and a further 40%, diagnosed at earlier stages with local disease only, eventually develop metastases (S. Vatandoust, T. J. Price, C. S. Karapetis, World J. Gastroenterol. 21, 11767-11776 (2015)). CRC metastases are hardly curable, with 13.5% of metastatic (m)CRC patients surviving 5 years post standard of care treatment initiation (J. Ferlay, I. et al. Int. J. Cancer. 136, E359-386 (2015)). The immune microenvironment of primary colon tumors is among the central determinants of disease outcome. Indeed, the Immunoscore, which recapitulates the type, location and density of primary CRC-infiltrating effector CD8³⁰ T cells, is correlated to better relapse-free survival (J. Galon, et al. Science. 313, 1960-1964 (2006); F. Pagès, B. et al. Lancet Lond. Engl. 391, 2128-2139 (2018)). Additional lymphoid or myeloid cell populations, including regulatory CD4⁺ T cells (Treg), CD4³⁰ Th17 cells, macrophages or myeloid-derived suppressor cells (MDSC), can promote or impede the effector immune response, although their prognostic value has not been firmly established in mCRC (M. Schmitt, F. R. Greten. Nat. Rev. Immunol. 21, 653-667 (2021); D. Bruni, H. K. Angell, J. Galon. Nat. Rev. Cancer. 20, 662-680 (2020)). Using a CRC syngeneic orthotopic mouse model, the inventors previously uncovered the ability of intra-colon (IC) tumors to promote opposite and mutually exclusive polarization of their microenvironment toward pro-or antitumor immune responses (G. Trimaglio, et al., Oncoimmunology. 9, 1790125 (2020)). The spontaneous antitumor polarization of orthotopic IC tumors, characterized by high proportions of tumor-infiltrating CD8⁺ T cells, leads to IC tumor regression (G. Trimaglio, et al., Oncoimmunology. 9, 1790125 (2020)). While targeting directly CD8⁺ T cells, efficacy of immunotherapy by immune checkpoint blockade (ICB), which led to clinical benefit in several malignancies (T. F. Gajewski, T. Schumacher, Cancer immunotherapy. Curr. Opin. Immunol. 25, 259-260 (2013)), also relies on the overall immune microenvironment (J. M. Pitt, M et al. Immunity. 44, 1255-1269 (2016); M. M. Gubin, E et al. Cell. 175, 1443 (2018)).

Despite the impact of primary CRC immune microenvironment on disease outcome, mCRC remains largely unresponsive to ICB (S. L. Topalian, et al. N. Engl. J. Med. 366, 2443-2454 (2012); C. Eng, et al. Lancet Oncol. 20, 849-861 (2019)). The specific liver localization of CRC metastases was shown to participate in restraining ICB efficacy (J. Yu, M et al. Nat. Med. 27, 152-164 (2021)).

Thus, it remains a need to develop a method for determining the potential of mCRC patient to respond to an immunotherapy.

The gut is a unique barrier site which continuously sustains effector and suppressive immune processes in order to tolerate microbiota and dietary antigens, to control pathogens and to maintain homeostasis (S. Shalapour, et al. Annu. Rev. Immunol. 38, 649-671 (2020)). Gut immune responses involve distinct components, including key lymphoid populations, such as effector CD8⁺ T cells and tolerogenic CD4⁺ T cells, that express the gut-homing α4β7 integrin (A. Habtezion, et al. Gastroenterology. 150, 340-354 (2016)). Although gut immunity develops locally, it may impact systemic extra-intestinal sites. For instance, gut and liver immunity are connected through the so-called gut-liver axis, which refers to bidirectional relationship between gut microbiota and the liver (A. Albillos, A. et al. J. Hepatol. 72, 558-577 (2020)). Translocation of gut-specific antigens toward the liver, through portal circulation, has been demonstrated to participate in the regulation of liver immunity, leading to shared memory T-cell responses to mucosal antigens (D. H. Adams, et al. Nat. Rev. Immunol. 6, 244-251 (2006)). In addition, a role for pathological gut-derived α4β7⁺CD8⁺ T cells is evoked in the development of inflammatory bowel disease (IBD)-associated hepatic disorders (D. H. Adamset al. Nat. Rev. Immunol. 6, 244-251 (2006); B. Eksteen, et al. J. Exp. Med. 200, 1511-1517 (2004)), inferring that gut-derived immune cells could be recruited to extra-intestinal sites, albeit this concept needs further validation.

In a proof-of-concept study, the inventors previously unveiled that immune communication between two concomitant tumors, located at different sites, could impact tumor outcome. Indeed, preclinical orthotopic renal tumors were able to modulate the immune microenvironment of distant concomitant lesions (C. Devaud, et al. Oncoimmunology. 4, e975572 (2015)).

### SUMMARY OF THE INVENTION

Herein, the inventors investigated whether gut-derived immune responses to primary colon tumors could be involved in systemic immunosurveillance of extra-intestinal metastases. They showed that colon tumors exert an immune-dependent systemic antimetastatic effect able to impact distant metastases. This antimetastatic effect is predictable by a MicroEnvironment score (ME-score).

The present invention relates to a method for determining the response of a metastasis to immunotherapy in a mCRC patient, said method comprising:
i) determining the density of CD8⁺ T cells, preferably enterotropic CD8⁺ T cells, more preferably α4β7⁺CD8⁺ T cells, dendritic cells, and macrophages in a patient sample,
ii) calculating a MicroEnvironment score (ME-score) corresponding to the ratio of the density of CD8⁺ T cells and dendritic cells to the density of macrophages, wherein a higher value of the ME-score in comparison to a control value is indicative that the metastasis of the patient is responsive to immunotherapy.

In a preferred embodiment, said method further comprises:
i) determining the density of B-cells, NK cells and/or regulatory T cells in a patient sample,
ii) calculating a MicroEnvironment score (ME-score) based on the ratio of density of CD8⁺ T cells, dendritic cells, B cells and/or NK cells on the density of macrophages and preferably regulatory T cells.

In the present application, the inventors showed, in multi-tumor mouse models and in mCRC patients, that enterotropic tumor-specific α4β7³⁰ CD8⁺ effector T cells impact both the fate of distant extra-intestinal tumors and their response to immunotherapy.

The present invention also relates to a method for determining the response of a metastasis to immunotherapy in a metastatic colorectal cancer patient, said method comprising determining the density of enterotropic CD8⁺ T cells, preferably α4β7⁺ CD8⁺ T cells in a patient sample, wherein a higher density of said enterotropic CD8⁺ T cells, preferably α4β7⁺ CD8⁺ T cells in comparison to a control value is indicative that the metastasis of the patient is responsive to immunotherapy.

In a preferred embodiment, according to the methods of the present disclosure said dendritic cells (DC) are CD11c⁺ cells and/or said macrophages are CD68⁺ cells at a protein level.

According to the methods of the present invention, the density of cells is preferably determined by flow cytometry, immunofluorescence, immunohistochemistry, bulk RNA-seq, qRT-PCR or single cell RNA-Seq. In a preferred embodiment, said patient sample is tumor tissue or biological fluid sample, preferably metastatic tumor tissue or patient blood sample such as patient peripheral blood mononuclear cells.

In another aspect, the present invention relates to an immunotherapy for use in the treatment of a metastatic colorectal cancer in a patient in need thereof wherein said immunotherapy is administered in said patient previously identified as having metastasis responsive to immunotherapy in a method as described above.

The present invention also relates to a pharmaceutical composition comprising an immune-stimulatory agent for use in the treatment of a metastatic colorectal cancer in a patient in need thereof wherein said immune-stimulatory agent is administered in said patient previously identified as having metastasis non-responsive to immunotherapy in a method as described above. Preferably said immune-stimulatory agent is a colorectal cancer vaccine, preferably able to elicit tumor-antigen specific α4β7⁺CD8⁺ T cells. In a preferred embodiment, said pharmaceutical composition comprises retinoic acid.

In another aspect, the present invention relates to a pharmaceutical composition comprising an immune-stimulatory agent, preferably a colorectal cancer vaccine and retinoic acid for use in the treatment of a metastatic colorectal cancer in a patient in need thereof.

The present invention also relates to a kit for determining the response of a metastasis to immunotherapy in a metastatic colorectal cancer patient comprising anti-CD8 antibody, preferably anti-α4β7 and anti-CD8 antibodies, preferably anti-CD8, anti-CD11c and/or anti-CD68 antibodies, more preferably anti-α4β7, anti-CD8, anti-CD11c and/or anti-CD68 antibodies or a kit for determining the response of a metastasis to immunotherapy in a metastatic colorectal cancer patient comprising a pair of primers for amplifying at least one gene surrogate marker of CD8⁺ T cells including CD8A and/or CD8B, preferably at least one gene marker of CD8⁺ T cells and at least one gene marker of α4β7⁺ cells including ITGA4 and ITGB7, more preferably at least one gene marker of CD8⁺ T including CD8A and/or CD8B, at least one gene marker of dendritic cell including ITGAX and at least one gene marker of macrophages including CD68, again more preferably at least one gene marker of CD8⁺ T cells and at least one gene marker of α4β7⁺ cells, at least one gene marker of dendritic cell and at least one gene marker of macrophages.

### DETAILED DESCRIPTION OF THE INVENTION

The terms “subject” and “patient” are used interchangeably herein and refer to both human and non-human animals. As used herein, the term “patient” denotes a mammal, such as a rodent, a feline, a canine, and a primate. Preferably, a patient according to the invention is a human, preferably a human metastatic cancer patient, preferably a human metastatic colorectal cancer patient.

The terms “cancer”, “tumor”, are used interchangeably herein to refer to cells that exhibit relatively abnormal, uncontrolled, and/or autonomous growth, so that they exhibit an aberrant growth phenotype characterized by a significant loss of control of cell proliferation.

The term “metastatic cancer”, “metastatic tumor or “metastasis” are used interchangeably herein to refer to a secondary tumor that is the result of the invasion of cancer cells through the bloodstream or lymphatic vessels to other sites and tissues in the body.

As used herein, the term “colorectal cancer” or “CRC” is understood to refer to bowel cancer. This term includes colon and rectal cancer in which malignant cells form in the tissues of the colon or the rectum. Colorectal cancer often begins as small, noncancerous clumps of cells called polyps that form on the inside of the colon or rectum and over time some of these polyps can become colon cancers. Cancer cells may break away from a tumor in the colon or rectum and spread to other parts of the body through the bloodstream or the lymphatic system. So colorectal cancer that spreads, or metastasizes, to the lungs, liver or any other organ is called metastatic colorectal cancer. The most common site of metastases for colorectal cancer is the liver, but colorectal cancer cells may also spread to any other organs such as non-limiting examples the lungs, bones, brain or spinal cord.

The term “patient sample” means any biological sample derived from a patient. Examples of such samples include tissue sample, biological fluid sample, cell samples, organs, biopsies. In a preferred embodiment, said patient sample is a tumor tissue such as metastatic tumor tissue or biological fluid sample such as blood patient sample, preferably peripheral blood mononuclear cells.

### The method for Determining the Response of a Metastasis to Immunotherapy in a Metastatic Colorectal Cancer Patient

In the present application, the inventors showed that a higher value of the ME-score calculated based on the ratio of the density of CD8⁺ T cells and dendritic cells to the density of macrophages in comparison to a control value is indicative that the metastasis of a metastatic colorectal cancer patient is responsive to immunotherapy.

According to the present disclosure, the terms “determining the response of a metastasis to a treatment”, or “determining metastasis responsiveness to a treatment” refers to an ability to assess whether the treatment (e.g., immunotherapy) is effective in (e.g., providing a measurable benefit or positive medical response to) the metastasis of the patient before and/or after some time of administration of the treatment. In another terms, according to the present disclosure, determining the response to immunotherapy refers to an ability to assess whether a metastasis is responsive to immunotherapy and for example whether following the immunotherapy the number of metastatic cancer cells or the size of a metastatic tumor is reduced, the progression of a metastatic cancer to a more aggressive form (i.e. maintaining the cancer in a form that is susceptible to a therapeutic agent) is reduced, the proliferation of metastatic cancer cells or of the speed of metastatic tumor growth are reduced, metastatic cancer cells are killed or the likelihood of recurrence of a metastatic cancer is reduced in a subject.

The present disclosure relates to a method for determining a response of a metastasis to an immunotherapy in a metastatic colorectal cancer patient.

Immunotherapy is a type of cancer treatment that activates the immune system to fight disease such as cancer. Immunotherapy that can be used to treat cancer includes as non-limiting examples: immune inhibitory checkpoint inhibitors which are drugs that block inhibitory immune checkpoint protein, T-cell transfer therapy, monoclonal antibodies or immune system activators.

As used herein the term “immune checkpoint protein” has its general meaning in the art and refers to a molecule that is expressed by T cells and NK cells and regulates the immune system. According to the present disclosure, immune checkpoint proteins are preferably inhibitory immune checkpoint proteins that dampen effector immune response. Inhibitory immune checkpoint molecules are recognized in the art to constitute immune checkpoint pathways similar to the CTLA-4 and PD-1 dependent pathways (see e.g., Pardoll, 2012. Nature Rev Cancer 12:252-264; Mellman et al., 2011. Nature 480:480-489).

As used herein, the term “inhibitory immune checkpoint inhibitor” or “immune checkpoint inhibitor” has its general meaning in the art and refers to any compound inhibiting the function of an immune inhibitory checkpoint protein. Inhibition includes reduction of function and full blockade. In particular, the immune checkpoint inhibitor particularly suitable for enhancing the proliferation, migration, persistence and/or cytotoxic activity of CD8⁺ T cells in the patient and in particular the tumor-infiltrating of CD8⁺ T cells of the patient.

In an embodiment, said immune checkpoint inhibitor of immune cells (T and B lymphocytes) is selected from the group consisting of anti-PD-L1, anti-PD-1, anti-CTLA-4, anti-HVEM, anti-BTLA, anti-TIGIT, anti-TIM-⅓, anti-LAG-3, and anti-OX40 agonist, anti-CD40 agonist, CD40-L, TLR agonists, and B-cell receptor agonists, in particular selected from the group consisting of anti-PD-L1, anti-PD-1 and anti-CTLA-4. In a particular embodiment of the disclosure, the immune checkpoint inhibitor is an anti-PD-L1 antibody.

In a particular embodiment, examples of immune checkpoint inhibitors are inhibitors that affect the PD-1/PDL-1 and CTLA-4 pathways and can be selected from the group consisting of: Ipilimumab, Nivolumab, Pembrolizumab, Atezolizumab, Avelumab and durvalumab.

According to the present disclosure said immunotherapy can be T cell transfer therapy. As used herein, T-cell transfer therapy, also called adoptive cell therapy, adoptive immunotherapy, or immune cell therapy has its general meaning in the art and refers to a treatment that boosts the natural ability of T cells to fight cancer. In this treatment, immune cells are taken from the patient tumor. T-cell transfer therapy can be tumor infiltrating lymphocytes or CAR T-cell therapy.

According to the present disclosure, immunotherapy can be monoclonal antibodies that binds to specific targets on cancer cells such as anti-CD20, anti-HER2, anti-EGFR, anti-VEGF, anti-CD52 or anti-CD33 or immune system activators such as cytokines including as non-limiting examples: interferon alpha (IFN-α), interleukin-2 (IL-2), interleukin-11 (IL-11), interleukin-21 (IL-21), erythropoietin, granulocyte-macrophage colony-stimulating factor (GM-CSF) or granulocyte colony-stimulating factor (G-CSF).

The present disclosure relates to a method for determining the response of a metastasis to immunotherapy in a metastatic colorectal cancer patient, said method comprising:
i) determining the density of CD8⁺ T cells, dendritic cells and macrophages in a patient sample,
ii) calculating a MicroEnvironment score (ME-score) corresponding to the ratio of the density of CD8⁺ T cells and dendritic cells to the density of macrophages, wherein a higher ME-Score is indicative that the metastasis of the patient is responsive to immunotherapy.

In a preferred embodiment said CD8⁺ T cells are enterotropic CD8⁺ T cells. Within the CD8⁺ T-cell population, which was one of the ME-score components, enterotropic cells expressing α4β7 integrin were key players of the colon antimetastatic effect. Enterotropism is the tropism toward intestine and/or gut. In a more preferred embodiment, said CD8⁺ T cells are α4β7⁺ CD8⁺ T cells.

Integrin α4β7 is described as a gut-tropic molecule as it is only acquired by T cells when activated by intestinal dendritic cells in mesenteric lymph node. It binds Mucosal Addressing Cell Adhesion Molecule-1 (MAdCAM-1), which is constitutively expressed on high endothelial venules (HEV) of Gut Associated Lymphoid Tissues (GALT), including Peyer patches and mesenteric (m)LN, as well as on postcapillary venules of the gut lamina propria.

In a preferred embodiment, the method according to the present disclosure further comprises:
i) determining the density of B-cells and/or NK cells in a patient sample and
ii) calculating a MicroEnvironment score (ME-score) corresponding to the ratio of the density of CD8⁺ T cells, dendritic cells, B-cells and/or NK-cells to the density of macrophages,
   wherein a higher ME-Score is indicative that the metastasis of the patient is responsive to immunotherapy.

In a more preferred embodiment, the method according to the present disclosure further comprises:
i) determining the density of regulatory T cells in a patient sample and
ii) calculating a MicroEnvironment score (ME-score) corresponding to the ratio of the density of CD8⁺ T cells and dendritic cells to the density of macrophages and regulatory T cells,
   wherein a higher ME-Score is indicative that the metastasis of the patient is responsive to immunotherapy.

In a another more preferred embodiment, the method according to the present disclosure further comprises:
i) determining the density of B-cells, NK cells and regulatory T cells in a patient sample and,
ii) calculating a MicroEnvironment score (ME-score) corresponding to the ratio of the density of CD8⁺ T cells, dendritic cells, B-cells and/or NK-cells to the density of macrophages and regulatory T cells,
   wherein a higher ME-Score is indicative that the metastasis of the patient is responsive to immunotherapy.

The method according to the present disclosure comprises the step of determining the density of said cells in a patient sample.

The cell densities can be evaluated by determining the number of each cell type in a patient sample by using specific cell markers. Specific cell markers are well known in the art and are used to distinguish one cell type, for example T cells, B cells, NK cells, dendritic cells, regulatory T cells or macrophages from other cells. Said specific cell markers can be a cell surface protein (also called herein cell surface antigen), intracellular protein, or gene that is specifically expressed by said cell type. In a preferred embodiment, said gene is the transcript (e.g. mRNA) of a gene that is specifically expressed by said cell type.

Each cell type can be identified by using one or a combination of specific cell markers. Specific cell markers are well-known in the art and some examples are provided below.

As used herein, the term “hematopoietic cell” has its general meaning in the art and refers to cells derived from hematopoiesis including red blood cells, lymphocytes and myeloid cells. Surface antigen marker for hematopoietic cells that can be used for detecting hematopoietic cells are well-known in the art and can be for example cell surface antigen CD45. Several antibodies that specifically bind to CD45 have been described in the prior art and are commercially available.

As used herein, the term “T cell” has its general meaning in the art and includes cells within the T cell lineage, including thymocytes, immature T cells, mature T cells and the like. According to the present disclosure, said T cell is a CD8⁺ T cell. In a preferred embodiment, CD8 surface antigen is detected by one of the several antibodies that specifically bind to CD8 that have been described in the prior art and are commercially available. In another embodiment, said CD8⁺ T cells can be identified by detecting at least one gene marker, preferably involved in CD8⁺ T cell activation and/or effector cytotoxic functions selected as non-limiting examples from the group consisting of: CD8A, CD8B, AHNAK, CX3Cr1, GZMM, CCL5, ITGBl, GZMB, ID2, CD226, CXCR3, TNFSFl, PRFl, GZMA and CCL3, preferably CD8A (CD8A isoforms, NCBI references NM_001145873.1, NM_001382698.1, NM_171827.4 or NM_001768.7, all last modified on Jun. 12, 2022) and CD8B (CD8B isoforms, NCBI references NM_004931.5 last modified on Jan. 30, 2022; NM_172101.5 last modified on Apr. 17, 2022; NM_172102.5 last modified on Apr. 17, 2022; NM_172213.5 last modified on Apr. 17, 2022 or NM_001178100.2, last modified on Jan. 30, 2022).

In a preferred embodiment said T cells is a α4β7⁺ CD8⁺ T cells. In a preferred embodiment, said α4β7⁺ cells are identified by using an anti-α4β7 antibody well-known in the art or by detecting ITGA4 (ITGA4 transcript variant 1 or 2, NCBI references NM_000885.6 last modified on Jun. 12, 2022 and NM_001316312.2 last modified on Jun. 12, 2022 respectively), ITGB7 (ITGB7 transcript variant 1, NCBI references NM 000889.3 last modified on Jun. 2, 2022), (D) 8A, and/or (D8B mRNAs the main surrogate markers α4β7⁺ CD8⁺ T cells at a transcriptomic level.

As used herein, the term “Regulatory T cell”, “Treg” formerly known as suppressor T cells has its general meaning in the art and includes subpopulation of T cells that modulates the immune system, maintain tolerance to self-antigens and prevent auto-immune disease. Treg can be distinguished from other cells by detecting surface antigen markers, for example selected in the group consisting of: CD4, FOXP3 and CD25 or a combination thereof. In another embodiment, said Treg can be identified by detecting at least one gene marker selected as non-limiting examples from the group consisting of: CD4, FOXP3 and CD25.

As used herein, the term “dendritic cell” has its general meaning in the art and refers to an antigen-presenting cells that present antigen on the cell surface to T-cells. Dendritic cells can be distinguished from other cells by detecting surface antigen markers, for example selected in the group consisting of: CD11c (ITGAX), HLA-DR, CLEC9A, CD141, CD1c or a combination thereof, preferably CD11c. Said surface antigens can be detected by one of the several antibodies that specifically bind to said surface antigens that have been described in the prior art and are commercially available. In a preferred embodiment said dendritic cells are conventional DCI dendritic cells and plasmacytoid dendritic cells. Said conventional DC1 dendritic cells can be distinguished from other cells by detecting surface antigen markers, for example selected in the group consisting of: CCR7, CD45RA, CD209, CLEC4C, LILRB4, NRP1, B220 and SiglecH or a combination thereof. Said plasmacytoid cells can be distinguished from other cells by detecting surface antigen markers, for example selected in the group consisting of: BLTA4, CAD1I, CD8A, CLEC9A, ITGAE, ITGAX, LY75, THBC and XCR1 or a combination thereof. In a particular embodiment said dendritic cell are identified by detecting mRNA of at least one gene marker selected from the group consisting of CLEC9A, SIGLECH, LY6C2, BST2, TCF4, IRF8, ITGAX and BcLllA, preferably ITGAX (ITGAX transcript variant 1 or 2, NCBI references NM_001286375.2 last modified on Jun. 6, 2022 and NCBI references NM_000887.5 last modified on Jun. 7, 2022 respectively). In a preferred embodiment, ITGAX is the main surrogate marker of DC at a transcriptomic level.

As used herein, the term “macrophages” has its general meaning in the art and refers to a cell that has the ability to phagocytic activity. Surface antigen marker that can be used for distinguishing macrophages from other cell types are well-known in the art and can be selected as non-limiting examples in the group consisting of: CD80, CD86, CCR5, CD11b, CD11c, CD14, EMRI, CD1la, CD33, CD15, CD68, CD163, CD64, CD32, CD16, CD16/32, CD115, CD369 (Dectin-1), CD204, CD206, CD209, FceR1, VSIG4, or a combination thereof, preferably CD68. In a preferred embodiment, said macrophages are immunosuppressive type 2 macrophages. In a particular embodiment, said macrophages are identified by detecting mRNA of at least one gene marker selected from the group consisting of TGM2, CHIL3, FN1, TGFB1, TGFB1, ARG1, IL10, CD163, CD206, IL-1 decoy R, IL-IRA, CD68 and MMPl2, preferably (CD68 (CD68 transcript variant 1 or 2, NCBI references NM_001251.3 last modified on Jan. 23, 2022 and NCBI references NM_001040059.2 last modified on Jan. 23, 2022 respectively). In a particular embodiment, said macrophages are immunosuppressive type 2 macrophages M2a, M2b, M2c or M2d macrophages. In a particular embodiment, said M2a macrophages can be identified by detecting mRNA of at least one gene marker selected from the group consisting of: CD163, MHCII, CD206, CD200R, TGM2, DecoyR, IL10, TFGB, IL1ra, CCL17, CCL22 and CCL24, said M2b macrophages can be identified by detecting mRNA of at least one gene marker selected from the group consisting of: CD86, MHCII, IL1, IL6, IL10, INFalpha, and CCL1, said M2c macrophages can be identified by detecting mRNA of at least one gene marker selected from the group consisting of: CD163, TLR1, TLR8, IL10, TGFbeta, and CCR2 and M2d macrophages can be identified by detecting mRNA of at least one gene marker selected from the group consisting of: VEGF, IL10, ILl2, TNFalpha, TGFB, CCL5, CXCL10 and CXCL15. In a preferred embodiment, CD68 is the main surrogate marker of macrophages at a transcriptomic level.

As used herein, the term “natural killer cells” or “NK cells” has its general meaning in the art and refers to cytotoxic lymphocytes critical to the innate immune system with both intrinsic cytotoxic potential and cytokine-producing capabilities. Surface antigen markers for NK cells that can be used for distinguishing NK cells from other cell types are well-known in the art and can be selected for example in the group consisting of: CD56, CD94, CD122, CD16, NKG2D, NKG2A, NKp30/44/46/80, KIR Family receptors or a combination thereof, preferably said marker is CD56. In a particular embodiment, said NK cells are identified by detecting mRNA of at least one gene marker selected from the group consisting of CD56, CD94, CD122, CD16, NKG2D, NKG2A, NKp30/44/46/80, KIR Family receptors, IFNG, PRF1, GZMA, PRF1, EOMES, NKG7, NCR1 and GZMB.

As used herein, the term “B cells” or “B lymphocytes” has its general meaning in the art and refers to immune cells that produce antibodies and generate immunological memory, function as antigen-presenting cells and secrete regulatory cytokines. Surface antigen markers for B cells that can be used for distinguishing B cells from other cell types are well-known in the art and can be selected as non-limiting examples in the group consisting of: CD19, B220 (CD45R), CD20 (memory B cells), IgD (follicular B cells), IgA, IgG and IgE (memory B cells), CD27 (memory and marginal zone B cells), CD38 in combination with CD138, CD78, IgG, CD27 (plasma cells), CD24 (regulatory B cells), CD25 and CD30 (activated B cells) or a combination thereof, preferably said marker is CD19. In a particular embodiment, said B-cells are identified by detecting mRNA of at least one gene marker selected from the group consisting of CD19, B220 (CD45R), CD20, IgD), IgA, IgG, IgF, CD27, CD38, CD138, CD78, CD24, CD25 and CD30.

In a particular embodiment, specific cell markers such as surface antigen markers as described above can be detected with an antibody such as a polyclonal antibody, monoclonal antibody, antigen-binding fragment or antibody mimetic using any well-known methods in the art such as immunofluorescence, immunohistochemistry or flow cytometry as described in the example 1.6 of the present application. The reactions generally include revealing labels such as fluorescent, chemiluminescent, radioactive, enzymatic labels or dye molecules, or other methods for detecting the formation of a complex between the antigen and the antibody or antibodies reacted therewith.

Several antibodies that specifically bind to said surface antigen as described above have been described in the prior art and are commercially available and for example are listed in antibody databank such as on-line databank Antibodypedia (Kiermer V. (2008) “Antibodypedia—A web portal to share antibody validation data” Nature Methods. 5: 860).

In another particular embodiment, the mRNA of gene markers as described above can be detected with a probe that hybridizes specifically to mRNA of gene markers using any well-known method in the art, for example in situ-hybridization.

In another particular embodiment, the gene markers as described above can be detected with primers designed for specifically amplifying the product of gene markers such mRNA or complementary DNA as described above, for example bulk RNA seq, qRT-PCR and single cell RNA seq as described in the example of the present application (Example 1.7).

According to the present disclosure, in a particular embodiment, cell density may be defined as the number of cells per one unit of cross-sectional area of metastatic tumor sample and can be determined for example by immunohistochemistry, immunofluorescence or in situ hybridization. Cell density may be expressed as the number of these cells that are counted per cm² or mm² of surface area of metastatic tumor patient sample or as the number of cells per one volume unit of sample, e.g. as the number of cells per cm³ of metastatic tumor tissue sample.

In another particular embodiment, the cell density may also be defined as the proportion of a cell type among the total cells, preferably among the total hematopoietic cells (e.g. CD45⁺) in a patient sample, preferably metastatic tumor patient sample or blood sample such as peripheral blood mononuclear cell and can be determined for example by flow cytometry or single cell RNA seq. In a particular embodiment, the ME-score can thus be calculated based on the ratio of the percentage of CD8⁺ T and dendritic cells among total hematopoietic cells (e.g. CD45⁺) to the percentage of macrophages among total hematopoietic cells (e.g. CD45⁺) in a tissue tumor sample or blood sample. In a preferred embodiment, said CD8⁺ T cells are enterotropic CD8⁺ T cells, more preferably α4β7⁺ CD8⁺ T cells.

In a preferred embodiment, the ME-score is calculated based on the ratio of the percentage of CD8⁺ T, dendritic, B-cell and/or NK cells among total hematopoietic cells (e.g., CD45⁺) to the percentage of macrophages and/or Regulatory T cells among total hematopoietic cells (e.g., CD45⁺).

In another particular embodiment, the cell density may also be defined by determining the total amount of surrogate marker gene transcripts in cell subject sample, in particular ITGA4,ITGB7, CD8A and/or CD8B as the main surrogate markers α4β7⁺ CD8⁺ T cells at a transcriptomic level, ITGAX and CD68 as the main surrogate markers of DC and macrophages respectively at a transcriptomic level, for example by bulk RNA seq or qRT-PCR. Indeed, the inventors have shown that the level of transcript of above-mentioned surrogate markers is proportional to the density of each cell type, and thus allow to deduce the cell density of CD8⁺ T cells, preferably α4β7⁺CD8⁺ T, DC and macrophages.

According to the present disclosure, the immunotherapy response of a metastasis in a metastatic colorectal cancer patient is evaluated by determining the ME-score in a patient sample and comparing said ME-score to a control value.

A high ME-score in a patient sample in comparison to a control value is indicative that the metastasis of the colorectal cancer patient is responsive to immunotherapy. A low ME-score in a patient sample in comparison to a control value is indicative that the metastasis of the colorectal cancer patient is non-responsive to immunotherapy.

The therapeutic response can be evaluated according to the present method, before immunotherapy or throughout the course of immunotherapy for monitoring the therapeutic response over time.

The inventors showed a central role for α4β7 CD8⁺ T cells which when migrating to metastases mediate metastatic tumor elimination. They showed that a higher density of α4β7 CD8⁺ T cells in tumor or blood sample in a metastatic colorectal cancer patient compared to a control value is indicative that the metastasis of the patient is responsive to immunotherapy.

The present application also relates to a method for determining the response of a metastasis to immunotherapy in a metastatic colorectal cancer patient, said method comprising determining the density of α4β7 CD8⁺ T cells in a patient sample, preferably patient tumor sample or biological fluid sample, preferably blood cell sample such peripheral blood mononuclear cells, wherein a higher density of said α4β7⁺CD8⁺ T cells compared to a control value is indicative that the metastasis of the patient is responsive to immunotherapy. The density of α4β7⁺ CD8⁺ T cells can be preferably determined as described above, for example by immunohistochemistry, immunofluorescence, flow cytometry or single cell or bulk RNA seq and qRT-PCR.

In a particular embodiment, the density of α4β7⁺ CD8⁺ T cells may be measured by determining the number of α4β7⁺ CD8⁺ T cells in a cross-sectional area of metastatic tumor sample, for example by immunohistochemistry or immunofluorescence as described above. In another particular embodiment, the density of α4β7⁺ CD8⁺ T cells is measured by determining the % of α4β7⁺ CD8⁺ T cells among total cells in a patient sample such as tumor tissue sample (e.g. metastatic tumor tissue) or biological fluid sample such as blood sample (e.g. peripheral blood mononuclear cells), preferably by determining the percentage of α4β7⁺ CD8⁺ T cells among total hematopoietic cells (e.g. CD45⁺) in a patient sample as described above. In another embodiment, the density of α4β7⁺ CD8⁺ T cells may be measured by determining the expression level of ITGA4, ITGB7, CD8A and/or CD8B mRNAs for example by qRT-PCR or bulk RNA seq.

According to the present disclosure, the immunotherapy response of a metastasis in a metastatic colorectal cancer patient is evaluated by determining the density of α4β7⁺ CD8⁺ T cells in a patient sample and comparing said density to a control value.

A higher density of α4β7⁺ CD8⁺ T cells in a patient sample compared to a control value is indicative that the metastasis of the metastatic colorectal cancer patient is responsive to immunotherapy. A lower density of α4β7⁺ CD8⁺ T cells in a patient sample compared to a control value is indicative that the metastasis of the colorectal cancer patient is non-responsive to immunotherapy.

The therapeutic response can be evaluated according to the present method, before immunotherapy and/or throughout the course of immunotherapy for monitoring the therapeutic response over time.

According to the present disclosure, the terms “threshold value”, “control value” or “cut-off value” can be used interchangeably and can be determined experimentally, empirically, or theoretically. The control value refers to the ME-score or α4β7⁺ CD8⁺ T cell density in a biological sample obtained from a general population or from a selected population of subjects. For example, the general population may comprise apparently healthy subjects, such as individuals who have not previously had any sign or symptoms indicating the presence of cancer. The term “healthy subjects” as used herein refers to a population of subjects who do not suffer from any known condition, and in particular, who are not affected with any cancer.

In a preferred embodiment, the control value refers to the ME-score or α4β7⁺ CD8⁺ T cell density in a biological sample obtained from other source than the patient's data, for example cancer patients who is not responsive to immunotherapy or with poor prognosis (e.g., a short disease-free survival time).

The control value may be established based upon comparative measurements between metastatic colorectal cancer patients responsive to immunotherapy and metastatic colorectal cancer patients no responsive to immunotherapy and calculating the statistical significance between the ME-score or α4β7⁺ CD8⁺ T cell density of a collection of patient samples and the corresponding responsiveness to immunotherapy of the metastatic colorectal cancer patients from which samples derive. Said predetermined reference value (cut-off value) can consists of the value for which the highest statistical significance is calculated. The control value may also be established based upon comparative measurements between metastatic colorectal cancer patients with poor or good prognosis (e.g. a long disease-free survival time). Typically, the control value can be determined using a Receiver Operating Characteristic (ROC) curve based on experimental data. For example, after determining the ME-score or α4β7⁺ CD8⁺ T cell density in a group of reference, one can use algorithmic analysis for the statistic treatment of the measured values in samples to be tested, and thus obtain a classification standard having significance for sample classification. In a particular embodiment, Receiver operating characteristic (ROC) analysis was performed to calculate the ME-score or α4β7⁺ CD8⁺ T cell density using patient samples with known clinical status. The ME-score or α4β7⁺ CD8⁺ T cell density offering the highest sensitivity and specificity was selected as cut-off point. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

### Therapeutic Uses

In another aspect, the present disclosure relates to an immunotherapy for use in the treatment of a metastatic colorectal cancer in a patient in need thereof wherein said immunotherapy is administered to said patient previously identified as having a metastasis responsive to immunotherapy in a method as described above.

Immunotherapy that can be used for the present therapeutic use can be selected from the group consisting of: immune checkpoint inhibitors which are drugs that block inhibitory immune checkpoint protein, T-cell transfer therapy, monoclonal antibodies and immune system activators as described above.

In a particular embodiment, the present disclosure relates to a method of treating a metastatic colorectal cancer in a patient in need thereof, comprising:
i) determining the density of CD8⁺ T cells, dendritic cells and macrophages and preferably B-cells and/or NK cells and more preferably Regulatory T cells in a patient sample,
ii) calculating a MicroEnvironment score (ME-score) corresponding to the ratio of density of CD8⁺ T and dendritic cells and preferably B-cells and/or NK-cells to the density of macrophages and preferably Regulatory T cells, wherein a higher value of ME-score in comparison to a control value is indicative that the metastasis of the patient is responsive to immunotherapy,
iii) administering a therapeutically efficient amount of an immunotherapy in said patient previously identified as having metastasis responsive to immunotherapy.

In a preferred embodiment, said CD8⁺ T cells are enterotropic CD8⁺ T cells, preferably α4β7⁺ CD8⁺ cells.

In the context of the present disclosure, the term “treating” or “treatment”, as used herein, means reversing, alleviating, inhibiting the progress of, or preventing the disorder or condition to which such term applies, or reversing, alleviating, inhibiting the progress of, or preventing one or more symptoms of the disorder or condition to which such term applies. Such treatment aims at improving the clinical status of the animal or human patient, by eliminating or lowering the symptoms associated with cancers, in particular metastatic colorectal cancer.

As used herein, a “therapeutically effective amount” or an “effective amount” means the amount of a composition that, when administered to a subject for treating a state, disorder or condition is sufficient to effect a treatment. The therapeutically effective amount will vary depending on the composition, the disease and its severity and the age, weight, physical condition and responsiveness of the subject to be treated.

In another particular embodiment, the present disclosure relates to a method of treating a metastatic colorectal cancer in a patient in need thereof, comprising:
i) determining the density of α4β7⁺ CD8⁺ T cells in a patient sample, wherein a higher value of the density of α4β7⁺ CD8⁺ T in comparison to a control value is indicative that the metastasis of the patient is responsive to immunotherapy,
ii) administering a therapeutically efficient amount of an immunotherapy in said patient previously identified as having metastasis responsive to immunotherapy.

The present disclosure also relates to the use of immunotherapy in the manufacture of a medicament for the treatment of metastatic colorectal cancer in a patient in need thereof previously identified as having a metastasis responsive to immunotherapy in a method as described above.

In another embodiment, the present disclosure relates to a pharmaceutical composition comprising an immune-stimulating agent and preferably retinoic acid for use in the treatment of a metastatic colorectal cancer in a patient in need thereof wherein said immunotherapy is administered to said patient previously identified as having a metastasis non-responsive to immunotherapy in a method as described above.

The present disclosure relates to a method of treating a metastatic colorectal cancer in a patient in need thereof, comprising:
i) determining the density of CD8⁺ T cells, dendritic cells, and macrophages and preferably B-cells and/or NK cells and more preferably Treg in a patient sample,
ii) calculating a MicroEnvironment score (ME-score) corresponding to the ratio of density of CD8⁺ T and dendritic cells and preferably B-cells and/or NK-cells to the density of macrophages and preferably Treg, wherein a lower value of ME-score in comparison to a control value is indicative that the metastasis of the patient is non-responsive to immunotherapy,
iii) administering a therapeutically efficient amount of an immune-stimulating agent and preferably retinoic acid in said patient previously identified as having metastasis non-responsive to immunotherapy.

In a preferred embodiment, said CD8⁺ T cells are enterotropic CD8⁺ T cells, preferably α4β7⁺ CD8⁺ T cells.

In another particular embodiment, the present disclosure relates to a method of treating a metastatic colorectal cancer in a patient in need thereof, comprising:
i) determining the density of α4β7⁺ CD8⁺ T cells in a patient sample, wherein a lower value of the density of α4β7⁺ CD8⁺ T in comparison to a control value is indicative that the metastasis of the patient is non-responsive to immunotherapy,
ii) administering a therapeutically efficient amount of an immune-stimulating agent and preferably retinoic acid in said patient previously identified as having metastasis non-responsive to immunotherapy.

The present disclosure also relates to the use of a pharmaceutical composition comprising an immune-stimulating agent and preferably retinoic acid in the manufacture of a medicament for the treatment of metastatic colorectal cancer in a patient in need thereof previously identified as having a metastasis non-responsive to immunotherapy in a method as described above.

Immune-stimulatory agents are agents that can elicit tumor antigen specific α4β7⁺CD8⁺ T cells response. Immune-stimulatory agents according to the present disclosure may be antibodies that block immune inhibitory checkpoints or any molecule involved in immune suppression the blockage of which is capable of triggering the tumor-specific immune response by α4β7⁺CD8⁺ T cells such as antibodies targeting any components from the tumor microenvironment (immune cells) and the stroma (fibroblast, endothelial cells); agonist antibodies that stimulate directly or indirectly tumor specific α4β7⁺CD8⁺ T cells; adoptive cellular therapies based on the infusion of tumor-specific α4β7⁺CD8⁺ T cells, therapeutic cancer vaccines against tumor antigens including overexpressed, tissue differentiation, cancer-testis, oncofetal, oncogenic antigens and neoantigens).

Said vaccine according to the present disclosure can comprise immune adjuvants including TLR agonists (Poly-ICLC, MPL, CpG ODN, Imiquimod), DC-targeted monoclonal antibody (DEC205, Agonist CD40-specific), saponin-based adjuvant, GM-CSF, Tetanus or diphtheria toxoid, and/or delivery vehicles including emulsions, liposomes, virosomes and nanodiscs.

In a preferred embodiment said immune-stimulatory agent is a cancer vaccine

The vaccine can be based on formulations including protein or peptide-based formulations, anti-idiotype antibody-based formulations, heat shock protein-based formulation and nucleic acid (DNA or mRNA)-based formulation, cell-based formulation (whole tumor cells, antigen-loaded DC) and vector-based formulation (viral, bacterial). Viral vector-based vaccines include, but are not limited to, poxvirus-based, adenovirus-based, herpesvirus-based (e.g. type 1 Herpes simplex virus) and adeno-associated virus-based vaccines. When the vaccine is a poxvirus-based vaccine, the poxvirus can be any suitable poxvirus, such as avipox (e.g., fowlpox, canarypox, and pigeonpox) or orthopox (vaccinia (e.g., MVA, NYVAC and ProstVac), cowpox, camelpox, and monkeypox).

In a particular embodiment, said cancer vaccine comprises a nucleic acid sequence encoding a peptide, or a peptide derived from tumor-associated antigens, that are only expressed in cancer cells and not in normal cells and are presented only on the cancer cell's surface. Peptides derived from TAAs bound to human leukocyte antigen (HLA) and can be recognized by T cells initiating an anti-cancer immune response.

In a particular embodiment, said cancer vaccine can be a neoantigen cancer vaccine achieved by measuring peptide-specific cytotoxic T lymphocytes precursors in the patients' sample, screening for peptide-specific IgGs, followed by vaccination with CTL-reactive peptides.

In a particular embodiment, TAA that can be used according to the present disclosure to initiate anti-colorectal cancer immune response is selected in the group consisting of: CEA, MAGE, MUC1, Survivin, WT1, RNF43, TOMM34, FOXMI, MELK, HJURP, KOC1,KRAS, AIM2, HT001 and TAF1B, cypB, Ick, SART1-3, ART-4, Lck, WHS, HNR, MRP3, PAP, EZH2, CEA, PSCA, UBE, Her2/neu, PSA, CynB, EIF4EBP and WHCS2 as reviewed in Wagner S, Mullins CS, Linnebacher M. World J Gastroenterol. 2018. December 28;24(48):5418-5432. In a preferred embodiment said TAA is a neoantigen.

In a preferred embodiment, said stimulated tumor-specific α4β7⁺CD8⁺ T cells can be directed to the gut by administrating a gut-driver. In a preferred embodiment said gut driver is retinoic acid known to confers gut-tropism to effector T cells (Hammerschmidt et al, J Clin Invest, 2011 August; 121(8):3051-61). In addition to natural retinoic acid agonist, synthetic retinoic acid agonists can be used. Exemplary retinoic acid agonists include, but are not limited to, Ro 13-7410, Ro 19-0645, and N-retinoyl-D-glucosamine.

In the present application, the inventors showed that intestinal-initiated immune response involving α4β7⁺CD8⁺ T cells confers anti-metastatic effect. Thus, a pharmaceutical composition that elicits tumor antigen specific α4β7⁺CD8⁺ T cells response can be used for the treatment of metastatic colorectal cancer in a patient in need thereof.

The present disclosure relates to a pharmaceutical composition comprising an immune-stimulatory agent and retinoic acid for use in the treatment of metastatic colorectal cancer in a patient in need thereof.

The present disclosure also relates to a method of treating a metastatic colorectal cancer in a patient in need thereof, comprising administering a therapeutically efficient amount of an immune-stimulatory agent and retinoic acid in said patient.

The present disclosure relates to the use of a pharmaceutical composition comprising an immune-stimulatory agent and retinoic acid in the manufacture of a medicament for the treatment of metastatic colorectal cancer in a patient.

Said pharmaceutical composition can comprise one or more pharmaceutical acceptable excipient, diluent or carrier. As used herein, the term “pharmaceutically acceptable” means approved by a regulatory agency or recognized pharmacopeia such as European Pharmacopeia, for use in animals and/or humans. The term “excipient” refers to a diluent, adjuvant, carrier, or vehicle with which the therapeutic agent is administered.

Any suitable pharmaceutically acceptable carrier, diluent or excipient can be used in the preparation of a pharmaceutical composition (See e.g., Remington: The Science and Practice of Pharmacy, Alfonso R. Gennaro (Editor) Mack Publishing Company, April 1997). Pharmaceutical compositions are typically sterile and stable under the conditions of manufacture and storage. Pharmaceutical compositions may be formulated as solutions (e.g. saline, dextrose solution, or buffered solution, or other pharmaceutically acceptable sterile fluids), microemulsions, liposomes, or other ordered structure suitable to accommodate a high product concentration (e.g. microparticles or nanoparticles). The carrier may be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition.

The composition (e.g. immunotherapy, immune-stimulating agent, retinoic acid or pharmaceutical composition as described above) may be administered by any means known to those skilled in the art, including, without limitation, intravenously, orally, intra-tumoral, intra-lesional, intradermal, topical, intraperitoneal, intramuscular, parenteral, subcutaneous and topical administration. Thus, the composition may be formulated as an injectable, topical, or ingestible formulation. Administration of the composition to a subject in accordance with the present disclosure may exhibit beneficial effects in a dose-dependent manner. Thus, within broad limits, administration of larger quantities of the composition is expected to achieve increased beneficial biological effects than administration of a smaller amount. Moreover, efficacy is also contemplated at dosages below the level at which toxicity is seen.

It will be appreciated that the specific dosage of an administered in any given case will be adjusted in accordance with the composition (e.g. immunotherapy, immune-stimulating agent, retinoic acid or pharmaceutical composition as described above) being administered, the volume of the composition that can be effectively delivered to the site of administration, the disease to be treated or inhibited, the condition of the subject, and other relevant medical factors that may modify the activity of the compositions or the response of the subject, as is well known by those skilled in the art.

For example, the specific dose of composition (e.g. immunotherapy, immune-stimulating agent, retinoic acid or pharmaceutical composition as described above) for a particular subject depends on age, body weight, general state of health, diet, the timing and mode of administration, the rate of excretion, medicaments used in combination and the severity of the particular disorder to which the therapy is applied. Dosages for a given patient can be determined using conventional considerations, e.g., by customary comparison of the differential activities of the immunotherapy and pharmaceutical compositions described herein and of a known agent, such as by means of an appropriate conventional pharmacological protocol. The composition (e.g. immunotherapy, immune-stimulating agent, retinoic acid or pharmaceutical composition as described above) can be given in a single dose schedule, or in a multiple dose schedule.

Suitable dosage ranges for composition (e.g. immunotherapy, immune-stimulating agent, retinoic acid or pharmaceutical composition as described above) may be of the order of several hundred micrograms of the agent with a range from about 0.001 to 10 mg/kg, preferably with the range from about 0.01 to 1 mg/kg, more preferably from about 1 to 10 mg/kg, again more preferably 10 mg/kg.

### Kit

In another aspect, the present disclosure relates to a kit for determining the response of a metastasis to immunotherapy in metastatic colorectal cancer patient comprising a set of reagents that enables the determination of ME-Score.

In a particular embodiment, said kit comprises anti-CD8 antibody, preferably anti-α4β7 and anti-CD8, more preferably anti-CD8, anti-CD11c and anti-CD68 antibodies, again more preferably anti-α4β7, anti-CD8, anti-CD11c and anti-CD68 antibodies.

In another particular embodiment, said kit comprises a pair of primers for amplifying at least one gene marker of CD8⁺ T cells as described above including CD8A and/or CD8B, preferably a pair of primers for amplifying at least one gene marker of CD8⁺ α4β7⁺ T cells as described above including ITGA4, ITGB7 CD8A and/or CD8B, more preferably a pair of primers for amplifying at least one gene marker of CD8⁺ T cells as described above including CD8A and/or CD8B, a pair of primers for amplifying at least one gene marker of dendritic cell as described above including ITGAX and a pair of primers for amplifying at least one gene marker of macrophages as described above including CD68, again more preferably a pair of primers for amplifying at least one gene marker of CD8⁺ T cells as described above including CD8A and/or CD8B and at least one gene marker of α4β7⁺ T cells as described above including ITGA4 and/or ITGB7, a pair of primers for amplifying at least one gene marker of dendritic cell as described above including ITGAX and a pair of primers for amplifying at least one gene marker of macrophages as described above including CD68.

In a more preferred embodiment, said kit may further comprise a pair of primers for amplifying at least one gene marker of B and/or NK cells as described above.

In another particular embodiment, said kit can comprise a probe that hybridizes under standard low stringent conditions to at least one gene marker as described above.

Preferably, the kit comprises containers each comprising one or more compounds in a concentration or amount that facilitates reconstitution and/or use of a reagent set, preferably primers or antibodies, preferably antibodies coupled to a label, preferably a fluorescent (fluorophore) or chemiluminescent (chromophore) compound that allows the determination of the ME-score and implementation of the method according to the disclosure.

The kit may also include instructions indicating methods for preparing and/or using the reagents to determine the expression level of said genes according to the methods of the disclosure.

### FIGURE LEGENDS

FIG. 1: Systemic-antimetastatic effect of colon tumors. (A to C) B6 mice (n=6−19 mice, one experiment representative of two) (A, B) were injected intra-hepatic (IH) with MC38-Luc cells alone (IH alone, IH al.) or concomitantly (IH+IC) with intra-colon (IC) injections of MC38 cells. (A) Individual bioluminescent emissions of IH tumors and (B) representative photos of bioluminescence from one mouse per group from IH alone, progressive (IH+IC Progr.) and rejecting (IH+IC Rej.) groups. [Individual results are represented]. One experiment representative of two independent experiments is depicted. (C) B6 mice were injected subcutaneously (SC), on each flank, with MC38 cells concomitantly (n=26) or not (n=6) with MC38-Luc cells IC. At day 10 post tumor-implantation, one SC-tumor was resected for future analyses and the remaining SC and IC tumor growths were monitored respectively using a caliper and the IVIS camera. Graph of SC tumors size (left axis) and IC tumors bioluminescent emission (right axis) depicts one representative mouse from progressive (Progr.), rejecting-relapse (Rej.-Rel.) and fully rejecting (Rej.) groups. Mann-Whitney statistical test was used: * P<0.05, **P<0.01, ***P<0.001.

FIG. 2: Microenvironment (ME)-score predicts the colon antimetastatic effect occurrence. (A to G) B6 mice were injected IH with MC38-Luc cells alone (IH alone, n=10) or concomitantly with IC injections of MC38 cells (IH+IC, n=33). Mice were monitored for IH tumors (tum.) bioluminescent emission from day (D) 2 to D9 and D10 IH and IC tumors were analyzed by flow cytometry. For each panel, one experiment representative of two is depicted. (A) ME-score established in individual IH tumors from IH+IC group, based on percentage (%) of CD8⁺ T cells (CD8) * % of dendritic cells (DC)/% of macrophages (MΦ). Light grey color represents the lowest ME-scores<25^{th} percentile (low; n=8), black color represents the intermediate ME-scores (Int.; n=17) and dark grey color represents the highest ME-scores>75^{th} percentile (High; n=8). Percentage of (B) CD8⁺ T cells, (C) DC, (D) MΦ, (E) PD-1⁺CD8⁺ T cells infiltrating IH or IC tumors (tum.) according to ME-score and in tumors from IH alone control group (IH al.) (F) Correlations between IH-tumor bioluminescent (biolum) emission and ME-score in IH+IC mice group (n=27) at D7; one point represents one mouse. (G) Individual IH-tumor bioluminescent emission in IH+IC mice at D2 and D9 according to ME-score (Low n=7; Int. n=13 and High n=7). (H) ME-scores in IH tumors from experiment depicted in previous panels (A to E) and from D10 dissociated-tumors following IH injections of TC1 (n=10), MOC1 (n=7), HEPA (n=9) and CT26 (n=10) in B6 and BALB/c mice. (I and J) B6 mice were injected subcutaneously (SC), on each flank, with MC38 cells alone (SC alone, n=6) or concomitantly with MC38-Luc cells IC (SC+IC, n=26). At D10, one SC-tumor was resected and analyzed by flow cytometry and the remaining SC and IC tumor growths were monitored (as described for FIG. 1E). (I) ME-scores in SC tumors from experiment depicted in FIG. 1E and (J) from D10 dissociated-tumors following SC injection of MOC1 (n=7), HEPA (n=7) and CT26 (n=10) in B6 and BALB/c mice. Legends for panels A to E appear after panel E, for panels F to H after panel H and for panels I and J after panel J. Individual results are represented with median with interquartile range in (A), with min to max and median with interquartile range (boxes) in (B, C, D, E, I) and with median only in (H, J). Spearman test (F), Student T-test or Mann-Whitney tests (other panels) were used: *P<0.05, **P<0.01, ***P<0.001, ****P<0.0001.

FIG. 3: Involvement of systemic tumor-specific CD8⁺ T cell response in the colon antimetastatic effect. (A) B6 mice were injected IH with MC38-Luc concomitantly with IC injection of MC38 cells (n=20) or B16F10 (n=19). Graph represents survival of mice. (B to E) B6 mice were injected IH with MC38-Luc cells concomitantly with IC injections of MC38 cells. At day (D) 10 ME-score was determined in IH tumors. Tumors (tum.) and peripheral compartments (spleen, blood, IH tumor draining lymph node (LN IH) and IC tumor draining lymph node (LN IC)) were analyzed by flow cytometry. (B) Representative dot plots (Dextramer versus CD8 stainings) and (C) percentage of Adpgk-dextramer⁺ CD8 T cells (Dextra+) infiltrating IH or IC tumors from mice bearing low and high ME-scores IH tumors. [Individual results are represented with mean ±SEM]. (D) Correlations between ME-score and % of dextramer⁺ CD8⁺ T cells in IH and IC tumors from mice bearing low and high ME-scores IH tumors. (E) Correlations between % of dextramer⁺ CD8⁺ T cells in IC tumors, spleen, blood, LN IC, LN IH and % of dextramer⁺ CD8⁺ T cells in IH tumors. Results are from two independent experiments pooled (B, C IH tum.) or from one experiment representative of two independent experiments. On linear regression curves one point represents one mouse. Log-rank Mantel-cox statistical test (A), Mann-Whitney and Pearson statistical tests were used: *P<0.05, **P<0.01, ***P<0.001, ****P<0.0001.

FIG. 4. α4β7⁺CD8⁺ T cells are key components of the ME-score and the colon-antimetastatic effect. (A to G) ME-score was evaluated in day 10 IH tumors from IH+IC implanted mice (MC38-Luc IH and MC38 IC; n=59) and from IH alone control mice (n=10). A pool of seven IH tumors per group with low, intermediate (Int.) and high ME-scores (IH+IC group) and from control group was used to perform scRNAseq analyses. (A and C) Uniform manifold approximation and projection (UMAP) of (A) total CD45+ cells and of (C) clusters of CD8⁺ T (CD8_c #), conventional CD4⁺ T (Tconv_c #), regulatory CD4⁺ T (Treg), NK (NK_c #) and NKT (NKT_c #) cells infiltrating IH tumors are colored by (A) CD45⁺ identified cell types and by (C) T lymphocytes/NK identified clusters. Macrophages: mΦ; CD8⁺ T lymphocytes: CD8; monocytes: mono; conventional dendritic cells: cDC; plasmacytoid DC: pDC; neutrophils: neutro. (B and D) Percentage of (B) individual CD45⁺ cell subsets, and (D) identified clusters of CD8⁺ T lymphocytes, infiltrating IH MC38 Luc-tumors from the merged groups. Each bar of histogram represents one scRNAseq sample. (E) Normalized expression of Itga4 and Itgb7 in the seven clusters (c1 to c7) of CD8⁺ T cells. (F) Mean of normalized expression and (G) UMAP visualization of Itga4 or Itgb7 in clusters CD8_c1, _c2, _c3, NK_c1 and Treg in low and high ME-scores tumors (expression in normalized counts). (F) Number of cells detected (count) expressing each gene per cluster is represented by dot size and level of genes expression (normalized count) by grey color scale. (H to K) B6 mice were injected IH with MC38-Luc concomitantly with IC injection of MC38 cells. (H) Representative dot plots (Dextramer versus α4β7 integrin stainings) and (I) percentage of α4β7 integrin⁺among Adpgk-dextramer⁺CD8 T cells (Dextra⁺) infiltrating IH tumors. (J) Representative dot plots and (K) percentage of α4β7integrin⁺ among circulating CD8⁺ T cells. Results are from two independent experiments pooled (I, n=12; K, n=14). (L and M) B6 mice were injected IH with MC38-Luc concomitantly with IC injection of MC38 cells and treated with anti-α4β7 integrin antibody (Anti-α4β7, n=25) or PBS as control (Ctl, n=25) as described in Materials and Methods. (L) Survival of mice and (M) proportion of alive and dead mice at different time points. Likelihood-ratio test was used for single-cell gene expression (E), Log-rank Mantel-cox statistical test was used for survival analyses (L) and Student T-test or Mann-Whitney statistical tests were used for panels (H) and (J): *P<0.05, **P<0.01, ****P<0.0001.

FIG. 5. α4β7⁺CD8⁺ T cells from colon tumors confer a therapeutic advantage during immunotherapy. (A and B) B6 mice were injected IH with MC38-Luc cells alone (groups: IH al. (n=14); IH+αPD-L1 (n=26)) or concomitantly with IC injections of MC38 cells (groups: IH+IC (n=29); IH+IC+αPD-L1 (n=29)). A group of mice was injected IC alone with MC38-Luc cells (IC+αPD-L1 (n=14)). Anti-PD-L1 antibody (αPD-L1) was injected in depicted group at day (D) 12 and D15. (A) Proportion of mice in which IH metastases were rejected (Rej.) or progressed (Progr.) and (B) survival of mice are depicted. (C to F) (C) IC alone (al.)-rejecting-recipient (rec.) mice (n=20) were rechallenged, 46 days post-initial IC implantation and rejection, by IH-injection with MC38-Luc cells. In parallel, naive B6 mice (C and D; n=6) (Control: Ctl) were IH-injected with MC38-Luc cells. (C and E) Individual IH-bioluminescent emissions at (C) early timepoints and (E) long term, and (D) representative photos of one mouse per group of rechallenged mice. One experiment representative of two independent experiments is depicted. (F) At D84 post IH-rechallenge (Post rech.), IC alone rejecting-recipient mice were sacrificed and depicted organs analyzed by flow cytometry. Graphs for % of Adpgk-dextramer⁺ CD8⁺ T cells (Dextra⁺) infiltrating liver at site of IH-tumor (tum.) injection (scar post-rejection), spleen, blood and LN IH, post-rechallenge (Post rech.) or in compartments from low and high ME-score tumor-bearing mice. (G) Swimmer plot representing time before (in months) and after (in weeks) treatment (T0) for 17 mCRC patients included in analyses. Patients 1 to 10 are non-responders and 11 to 17 are responders. (H) Mean of normalized expression of ITGA4 and ITGB7 in non-responders (blue background) and responders (red background). Percentage of patient per group is represented by dot size and level of gene expression (transcripts per million) by purple color scale. (I) Progression free survival curves in the 17 mCRC patients. The median of ITG4 and ITGB7 genes level of expression in RNAseq analyses was used to determine the low (Lo.) and high (Hi.) ITG4 and ITGB7 groups. (J) Representative dot plots and (K) percentage of α4β7 integrin⁺ among circulating CD8⁺ T cells for mCRC patients before treatment (T0). (L) Overall survival curve of the mCRC patients. The median of the percentage of α4β7integrin⁺ among circulating CD8⁺ T cells was used to determine the low (Lo.) and high (Hi.) α4β7 CD8 groups. [Individual results are represented with mean ±SEM in C, F and K]. Log-rank Mantel-cox statistical test was used for panels (B), (I) and (L) and Mann-Whitney statistical test was used for panels (C), (F) and (K): * P<0.05, **P<0.01, ***P<0.001, ****P<0.0001.

### EXAMPLES

### 1. MATERIAL AND METHODS

### 1.1 Cell Culture, Reagents and Tumor Cell Lines

MC38 (parental and firefly-luciferase+ (Luc)) (C57BL/6 (B6) background colorectal carcinoma, RRID: CVCL_0A67), B16F10 (B6 melanoma, RRID: CVCL_0159), HEPA1-6 (B6 hepatocellular carcinoma, ATCC Cat #CRL-1830) and CT26 (BALB/c colorectal carcinoma, ATCC Cat #CRL-2638) cell lines were cultured in Dulbecco's Modified Eagle's Medium (DMEM) (Sigma, Cat #6429) supplemented with 10% fetal bovine serum (FBS; Gibco, Cat #10270106). MC38-Luc were generated as previously described (C. Devaud, et al. Cancer Immunol. Immunother. CII. 62, 1199-1210 (2013)). TC1 cells (B6 background, generated by HPV16 E6/E7 and c-H-ras retroviral transduction of lung epithelial cells) were kindly provided by Pr. T. C. Wu (Johns Hopkins Medical Institutions, Baltimore, MD) and cultured in Roswell Park Memorial Institute (RPMI)-1640 (Thermo Fisher Scientific, Cat #61870044) with 10% FBS, 1% Sodium Pyruvate Solution (Sigma, Cat #S8636-100 mM), MEM Non-Essential Amino Acids (NEAA) Solution 1×(from solution 100×, Thermo Fisher Scientifc, Cat #11140035), HEPES 10 mmol/L (Thermo Fisher Scientific, Cat #15630056). MOC1 (B6 Mouse Oral Carcinoma cell line, Kerafast Cat #EWL001) were cultured in Iscove's Modified Dulbecco's Medium (IMDM) (Sigma, Cat #13390) with 10% FBS, Insulin (5 mg/ml; Sigma, Cat #16634-50 mg), Hydrocortisone (0.08 μg/ml; Sigma,

Cat #H0135-1 mg), Epidermal Growth Factor (EGF, 0.01 μg/mlMillipore, Cat #01-107), 2% Penicilin-Streptomycin (Sigma, Cat #P0781-100ML), 1% L-Glutamine (Thermo Fisher Scientific, Cat #25030024). All cells were cultured at 37° C. and 5% CO2 for 2 to 6 passages and tested for mycoplasma.

### 1.2 Mice.

6 week-old female B6 (JRj) or BALB/c mice were purchased from Janvier Laboratory (Le Genest-Saint-Isle, France). Mice were housed under specific pathogen free conditions at Centre Régional d′Exploration Fonctionnelle et de Ressources Experimentales (CREFRE, Toulouse, France). All experimental protocols were approved by the regional Ethic Committee of Toulouse Biological Research Federation (C2EA-01, FRBT) and by the French Ministry for Higher Education and Research (permission number: 23093-2019112715157467). For the guidelines on animal welfare, we followed the European directive 2010/63/EU. Mice were used at 7 to 10 weeks of age.

### 1.3 Tumor Inoculation, Monitoring and Treatment.

Mice were inoculated with one or two concomitant tumors (one or two tumor models).

For the intra-hepatic (IH) tumor model (IH alone), anesthetized mice were IH-injected with 2.5×10⁵ MC38-Luc, 5.10⁴ TC1, 5×10⁶ MOC1, 2×10⁶ HEPA1-6 or 10⁶ CT26 cells, as previously described (C. Devaud, et al. Mol. Ther. J. Am. Soc. Gene Ther. 22, 18-27 (2014)). For the subcutaneous (SC) tumor model (SC alone), mice were inoculated in their right flank with 2×10⁶ MC38, 5×10⁶ MOC1, 2×10⁶ HEPA1-6 or 10⁶ CT26 cells. For the intra-kidney (IK) tumor model (IK alone), anesthetized mice were injected IK with 5×10⁵ MC38 cells as previously described (C. Devaud, et al. Mol. Ther. J. Am. Soc. Gene Ther. 22, 18-27 (2014)). For the intra-colon (IC) tumor model (IC alone), anesthetized mice were injected with 5×10⁵ MC38-Luc cells as previously described (G. Trimaglio, et al. Oncoimmunology. 9, 1790125 (2020), C. Devaud, et al. Cancer Immunol. Immunother. CII. 62, 1199-1210 (2013)). For the IH+IC two tumor model, IH injection was performed in anaesthetized mice with 2.5×10⁵ MC38-Luc or 5×10⁵ B16F10 cells simultaneously with IC injection of 5×10⁵ MC38 cells. For the SC+IC two tumor model, mice were inoculated SC in both right and left flanks with 2×10⁶ MC38 cells, simultaneously with IC injection of 5×10⁵ MC38-Luc cells (one SC tumor was harvested at day(D) 10 for analysis). For the IH+IK two tumor model, IH injection was performed with 2.5×10⁵ MC38 cells with concomitant IK injection of 5×10⁵ MC38 cells.

For rechallenge experiments, day 46 IH+IC or IC alone rejecting mice were rechallenged by IH injection of 2.5×10⁵ MC38-Luc cells. MC38-Luc IH and IC tumor progression was monitored weekly in vivo using the IVIS® Spectrum in vivo Imaging System (PerkinElmer), following intraperitoneal (IP) injection of 150 mg/kg of D-Luciferin (Oz Bioscience, Cat #LN10000). Quantitative analyses were performed using IVIS Living Image 4.7.3 software (PerkinElmer). Bioluminescent signal intensity was presented as average radiance (photons/sec/cm²/sr). SC tumor progression was monitored using a caliper and tumor size was calculated in mm². Mice were euthanized when reaching the endpoint time (defined in ethical protocols) in survival experiments, when the SC tumor size reached the ethically defined limit of 250 mm² or when necessary for tumor and tissue harvesting and analysis.

For alpha4 beta7 integrin (LPAM-1) blockade, 100 μl of anti-LPAM-1 (clone DATK32, BioXcell, Cat #BE0034) or 100 μl of phosphate-buffered saline (PBS) as control were injected IP post tumor-implantation, first daily (between D1 and D8) at 350 μg/dose, and then at 200 μg/dose every 3 days until D17. For immune checkpoint blockade therapy, 100 ug/dose of anti-PD-L1 (clone 10F.9G2, BioXcell, Cat #BE0101) or Rat IgG2b isotype control (clone LTF-2, BioXcell, Cat #BE0090) were injected IP at D12 and D15.

### 1.4 Metastatic (m) CRC Patient Cohort.

Seventeen patients with proficient mismatch repair (pMMR) or micro-satellite stable (MSS) mCRC were included in the cohort following written informed consent under the MINER protocol (NCT03514368) at the Cancer University Institute of Toulouse. The protocol received approval from the Institutional Review Board, the french National Drug Administration Agency (Agence Nationale de Sécurité du Médicament et des produits de santé, ANSM) and the patient's protection committee (Comitéde Protection de Personnes, CPP). Following two or more lines of standard therapy, patients received immunotherapy combined with other agents. Treatment consisted in oral tyrosine kinase inhibitors (TKI) with antiangiogenic activity combined with anti-PD-1/PD-L1 immunotherapy (12 patients); anti-CTLA-4 and anti-PD-L1 immunotherapy (2 patients) or anti-PD-L1 immunotherapy with other immunomodulatory agents (3 patients). Thirteen patients presented synchronic metastases (metastatic lesions were detected at the time of initial diagnosis) and 4 patients presented metachronic metastases (metastases diagnosed after initial diagnosis and surgical treatment of the primary tumor). Among the 17 patients, seven were responders to immunotherapy and 10 were non-responders. Responders were defined as patients showing objective response (OR) on at least one lesion by RECIST (Response Evaluation Criteria In Solid Tumors) 1.1 criteria or stable disease (SD) for 6 months or more. Metastases biopsies obtained before treatment initiation (baseline) were formalin-fixed paraffin-embedded (FFPE) or frozen and blood samples at baseline (T0) were used to prepare PBMC. Metastatic lesions were localized in the liver (13 patients), lung (2), lymph node (1) and peritoneum (1).

### 1.5 Cell Isolation and Cryopreservation.

For mice tumor processing, D10 tumors were harvested following mice sacrifice. Tumors were sliced in small pieces and digested for 40 min using the Tumor Dissociation kit (Miltenyi Biotech, Cat #130-096-730) at 37° C. with agitation followed by filtration through a 70 μM cell strainer. Spleen and lymph nodes (LN) were also harvested, following mice sacrifice, and organs were directly dissociated by filtration through a 70 μM cell strainer. Blood was harvested at the time of sacrifice by intracardiac puncture using 37G needle, 1 ml syringe and 100 μl of heparin (SIGMA Cat #9041-08-1). An Ammonium-Chloride-Potassium (ACK) buffer (BD Biosciences Cat #555899) erythrocyte lysis step (5 min at room temperature) was performed on spleen and blood cell suspensions. All cell suspensions were frozen in FBS, 10% Dimethyl sulfoxide (DMSO) (SIGMA Cat #D2650). For human blood samples, peripheral blood mononuclear cells (PBMC) were isolated by density gradient sedimentation using Ficoll-Paque (Cytiva, Cat #17144003). Blood cells were cryopreserved in FBS, 10% DMSO.

### 1.6 Flow Cytometry

For mice samples, cells were thawed and resuspended in PBS, 2.5% FBS (staining buffer), containing anti-mouse CD16/CD32 monoclonal antibodies (mAbs) (clone 2.4G2, BD Biosciences 553142, 1:100) and incubated 10 minutes on ice. For dead cell exclusion, cells were centrifuged and resuspended in PBS containing Fixable Viability Stain 700 (BD Biosciences 564997, 1:1000) or Stain 780 (BD Biosciences 565388, 1:1000) for 10 minutes, in the dark, at room temperature. Cells were then washed twice in PBS. For MHC I/peptide dextramer staining, cells were incubated with 20 μl of PBS with 5% FBS containing PE-labeled H-2 Db/ASMTNMELM dextramers (mutated peptide from Adpgk gene (26), Immudex) for 30 minutes, in the dark, at room temperature. No wash was performed before subsequent surface staining with mAbs when dextramers were used. For surface staining, cells were incubated with combinations of the following anti-mouse mAbs, as indicated, in staining buffer, for 20 minutes, in the dark, at 4° C.: CD45.2 (clone 104, BD Biosciences, 1:100) conjugated with FITC (553772), BUV396 (564616) or BV650 (740490), CD3 (clone 145-2C11, BD Biosciences, 1:100) BV786 (564379) or BUV496 (564661), TCR beta chain FITC (clone H57-597, BD Biosciences 553170, 1:100), CD8a (clone 53-6.7, BD Biosciences) BUV737 (564297, 1:300) or BUV496 (750024, 1:100), CD4 BUV496 (clone GK1.5, BD Biosciences 564667, 1:300), TIGIT PerCP-eF710 (clone GIGD7, ThermoFisher 46-9501-82, 1:75), TIM-3 BV421 (clone RMT3-23, BD Biosciences 747626, 1:100), PD-1 BUV396 (clone J43, BD Biosciences 744549, 1:75), CD25 BV650 (clone PC61, BD Biosciences 564021, 1:100), CD11b PE (clone M1/70, Thermofisher 12-0112-81, 1:200), F4/80 BV786 (clone BM8, Biolegend 123141, 1:100), CD11c BV650 (clone NA418, Biolegend 117339, 1:100), Gr1 (Ly6G/C) AF700 (clone RB6-8C5, Biolegend 108422, 1:200), CD80 PECF594 (clone 16-10A1, BD Bioscience 562504, 1:200), I-Ab PE/Cy7 (clone AF6-120.1, Biolegend 116419, 1:100), CD49d (α4 integrin) BV711 (clone 9C10, BD Biosciences 740672, 1:100), beta7 integrin BUV396 (clone M293, BD Biosciences 743793, 1:100), FR4 BV510 (clone 12A5, BD Biosciences 744120, 1:100), LPAM-1 (α4β7 integrin) APC (clone DATK32, Miltenyi Biotec 130-123-565, 1:100) and NK1.1 PeCF-594 (clone PK136, BD Biosciences 562964, 1:100). For intracellular/intranuclear staining, cells were permeabilized 30 minutes at 4° C. using Foxp3/Transcription Factor Staining Buffer Set (Thermo Fisher Scientific, Cat #00-5523-00) except following dextramer staining where cell were permeabilized using Fixation/Permeabilisation kit (BD Biosciences, Cat #554714) for 20 minutes at 4° C. Staining with mAbs directed against intracellular/intranuclear markers was then performed for 30 minutes at 4° C.: FOXP3 PE (clone FJK-16S, eBioscience 12-5773-82, 1:100) and CTLA-4 PeCy7 (clone UC10-4B9, eBioscience 25-1522-82, 1:75). Flow cytometric analyses were performed using BD LSR FortessaTM X20 (BD Biosciences) and data were analyzed using Flowlogic software (Miltenyi).

For human samples, CD8 T cells were enriched from thawed cells by positive magnetic selection (CD8 MicroBeads, human, Miltenyi Biotec, 130-045-201) using MS Columns (Miltenyi Biotec, 130-042-201). Dead cells were excluded using Fixable Viability Stain 700 (20 min 4° C. in PBS) and cells were stained with the following anti-human mAbs: CD3 BUV496 (clone UCH-T1, BD Biosciences 612940, 1:50), CD8 V500 (clone RPA-T8, BD Biosciences 560774, 1:100), CD197 PE-Vio770 (clone REA546, Miltenyi Biotec 130-117-396, 1:50), CD45RA BUV395 (clone HI100, BD Biosciences 740298, 1:25), LPAM-1 (α4 beta β7 integrin) Alexa Fluor 405 (clone Hu117, R&D systems FAB10078V-100UG, 1:20), CD279 BUV737 (clone EH12.1, BD Biosciences 612791,1:50), TIGIT PE (clone MBSA43,eBioscience 12-9500-42, 1:50), in PBS containing 5% FBS, for 15 minutes at 4° C.

### 1.7 Droplet-Based Single-Cell RNA-Sequencing and Single-Cell Gene Expression Analysis.

CD45.2³⁰ cells were enriched by positive selection from tumor cell suspension by magnetic cell sorting (Miltenyi Biotec, Cat #130-110-618). Cells from mice were pooled based on their ME-score (Four samples prepared with IH tumors with low, intermediate and high ME-score and from IH alone control group). 3′ gene expression single-cell libraries were generated using the Chromium Controller Instrument and Chromium Next GEM Single Cell 3′ Kit v3 and Dual Index TT Set A, according to the manufacturer's instructions (10× Genomics) starting with 16500 cells per sample pool. Library size and quality were confirmed on Fragment analyzer (Agilent). KAPA quantification kit for Illumina platforms (KAPA Biosystems, Roche) was used to quantify libraries. Samples were pooled in equimolar fashion. The libraries were sequenced on a NextSeq 550 (Illumina) in pair end sequencing 28 bp (read1)×90 bp (read2) and dual index 10 bp. CellRanger mkfastq was used to demultiplex raw base call files with more than 97% perfect barcode match and more than 91% Q30 bases. Subsequently, FASTQ files for the four samples were processed with CellRanger count, to align to mm10 reference and quantify genes. The four samples were analyzed in R with Seurat package. For each sample, barcode cells were kept when they express less than 10% of mitochondrial genes and between 1000 and 6000 of total genes. The four samples were then merged and RNA count was normalized with SCTransform method. Barcoded cells that did not express Ptprc (CD45) and that express simultaneously Trac, Trbc1 and Trbc2 (TCR constant chains) or Adgre1 (F4/80) or Ighd (BCR constant chain) were removed. Principle component analysis (PCA) was performed on 3000 genes with RunPCA function. A Uniform Manifold Approximation and Projection (UMAP) dimensional reduction was then performed on scaled data using the top 15 principal components from the PCA. FindNeighbors and FindClusters functions were used on the 15 principal components and 18 clusters were identified. To characterize each cluster, FindAllMarkers function was used and significant genes with an average log2 fold-change greater than 1 were used in ImmGen. From these analyses, 12 major lineages were identified: CD8⁺ and CD4⁺ T cells (Tconv (conventional) and Treg (regulatory)), NK cells, NKT cells, B cells, monocytes, macrophages, dendritic cells (DC: conventional type 1 (cDC1), conventional type 2 (cDC2) and plasmacytoid (pDC)), and neutrophils. To characterize more precisely the macrophage and monocyte cell subtypes, FindMarkers function was used to compare macrophage and monocyte clusters and identify differential expressed genes in the 6 macrophage clusters and the monocyte cluster. T lymphocytes, NKT and NK cells (T/NKT/NK cells) were isolated, from the previous dataset of the 12 major lineages, and expression data were re-scaled on this new T/NKT/NK cells dataset. As for all CD45 cells, a PCA was performed on the T/NKT/NK cells dataset, with RunPCA function and the top 8 principal components were used for the UMAP analysis and to find each cluster. Finally, 15 clusters were identified using specific markers, described in the results. To characterize in detail the clusters, FindMarkers function was used and each cluster was compared to other clusters from similar cell type (CD8 clusters compared between them, Tconv and Treg clusters compared between them and NK and NKT clusters compared between them). FindAllMarkers and FindMarkers functions were used with likelihood-ratio test for single-cell gene expression. Marker genes were visualized on UMAP with normalized data and differentially expressed genes on Heatmap with the mean of normalized count for each sample.

### 1.8 RNA Extraction and Bulk RNA Sequencing Data Analysis on Human Tissue Samples.

After disruption and homogenization of the fresh frozen (FF) tumor samples, RNA was extracted using the Qiagen RNeasy Plus Mini kit (Qiagen, Hilden, Germany, Cat #74134). Formalin-Fixed Paraffin-Embedded (FFPE) tissue blocks were sectioned to prepare unstained sections (5 μm thick) on glass slides for RNA extraction. For FFPE samples, after scraping the tumor tissue present on 15 slides with a razor blade, samples were de-paraffinized using the Qiagen Deparaffinization Solution (Qiagen, Germany, Cat #19093) and RNA was extracted using the Qiagen RNeasy FFPE kit (Qiagen, Hilden, Germany, Cat #73504). RNA yield and purity were measured photometrically with NanoDrop (Thermo Scientifc) and Qubit Fluorometer (Thermo Fisher Scientific). RNA quality was analyzed using the Fragment Analyzer System (Agilent). Total RNA libraries were prepared using Illumina stranded Total RNA prep ligation with ribo-zero plus kit (Illumina, 20040525) starting with an input amount of 200 ng total RNA. Library size and quality were confirmed on Fragment Analyzer System (Agilent). KAPA quantification kit for Illumina platforms (KAPA Biosystems, Roche) was used to quantify libraries. Samples were pooled in equimolar fashion. Ten pb double indexed libraries were sequenced on a NextSeq 550 instrument (Illumina) using 74 base-length read chemistry in paired-end mode. For each patient, FASTQ raw files were trimmed with Trimgalore then aligned to hg38 genome with STAR. MarkDuplicates (PicardTools) was used to remove PCR duplicates. Htseq-count (HTSeq) was used to quantify genes and counts were normalized in TPM (transcripts per kilobase per million).

### 1.9 Statistical Analyses

The sample size for animal studies were guided by previous murine studies in our laboratory. Tumor growth, flow cytometry and survival data were analyzed using GraphPad Prism software. For group comparison, Student T-test, Mann Whitney test were used following evaluation of normality using Shapiro-Wilk normality test. Spearman test was use for correlation curves analyses. Patient overall survival (OS) and progression free survival (PFS) were calculated from immunotherapy initiation. Survival curves were compared using the log-rank test (Mantel-Cox). P<0.05 was considered statistically significant for all assays, and individual P values are denoted as follows: *P<0.05, **P<0.01, ***P<0.001, ****P<0.0001. Statistical analyses of scRNA-seq data are described above in the corresponding Materials and Methods paragraph.

### 2. RESULTS

### 2.1 Colon Tumors Exert a Systemic Antimetastatic Effect Predictable by an Immune Microenvironment Score.

The inventors implanted mice with MC38-Luc cells intra-hepatic (IH), as the main metastatic location, concomitantly or not with MC38 IC (intra-colon) implantation of primary tumor. Tumor progression was monitored by measuring bioluminescent emission which is positively correlated to tumor size (G. Trimaglio, et al. Oncoimmunology. 9, 1790125 (2020), C. Devaud, et al. Cancer Immunol. Immunother. CII. 62, 1199-1210 (2013)).

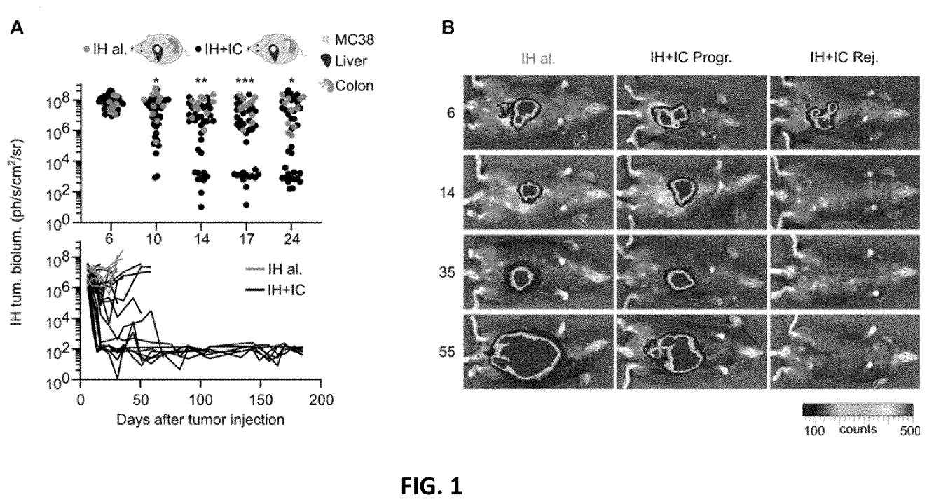

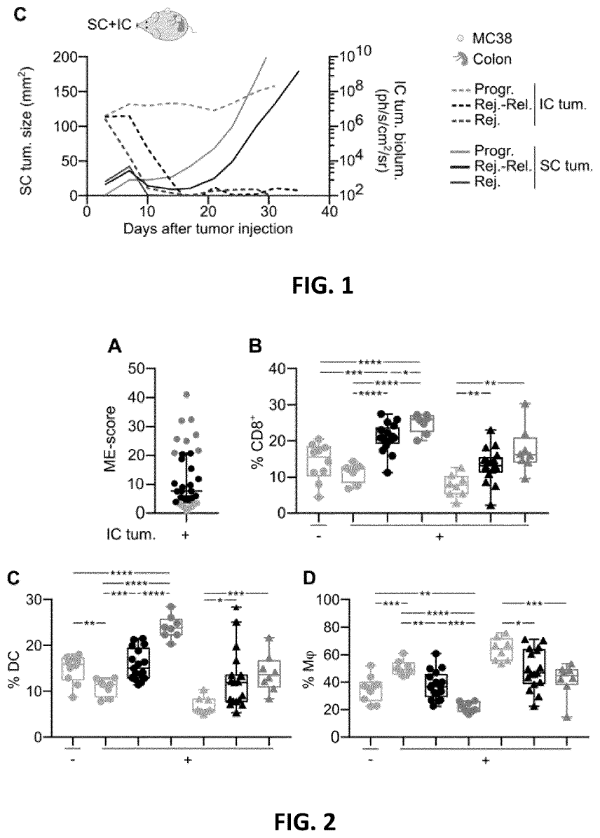

All mice implanted with IH tumors alone developed lethal hepatic tumors, whereas, when co-injected with IC tumors in addition to IH tumors (IH+IC group) and despite similar initial tumor take, half of mice rejected both their IH and IC tumors between days 8 and 14 (FIG. 1, A and B). IH tumor regression was observed only when concomitant IC tumors also regressed suggesting that IC-tumor rejection exerted an antimetastatic effect triggering distant hepatic tumor concomitant rejection. The inventors previously demonstrated that MC38-colon tumor rejection relies on antitumor immune response implying that the antimetastatic effect could be immune-dependent (G. Trimaglio, A.-F. et al. Oncoimmunology. 9, 1790125 (2020)). In addition, no IH tumor rejection was observed when the concomitant tumor was injected in another location than IC, such as intra-kidney (IK), suggesting that instatement of spontaneous tumor rejection and the ensuing antimetastatic effect were colon-specific.

To evaluate the broadness of the systemic colon antimetastatic effect, the inventors implanted MC38-tumors subcutaneously (SC), instead of IH, with or without concomitant IC tumors. All SC alone control mice developed lethal SC tumors. When injected concomitantly SC and IC, 66% of mice spontaneously rejected both their IC and SC tumors (FIG. 1C). Namely, when the IC tumor progressed, the SC tumor also progressed, whereas rejection of the IC tumor systematically impacted the distant SC tumor (FIG. 1C). However, contrarily to rejected IH tumors which did not recur over long-term follow-up periods (FIG. 1A), SC tumor rejection was long-lasting in 42% of mice whereas 24% of mice experienced relapse (FIG. 1C). These results further confirmed that the antimetastatic effect relied on IC tumor rejection and showed that it could also impact distant tumors in other locations than IH. The disparity between the IH and SC rejection profiles could infer that differences between the skin and liver immune microenvironments affect locally the colon-imprinted immune mechanisms underlying the systemic antimetastatic effect (J. M. Pitt, et al. Immunity. 44, 1255-1269 (2016)). Altogether, these results evidence an immune-dependent, systemic, colon-specific antimetastatic effect, able to impact distant tumors.

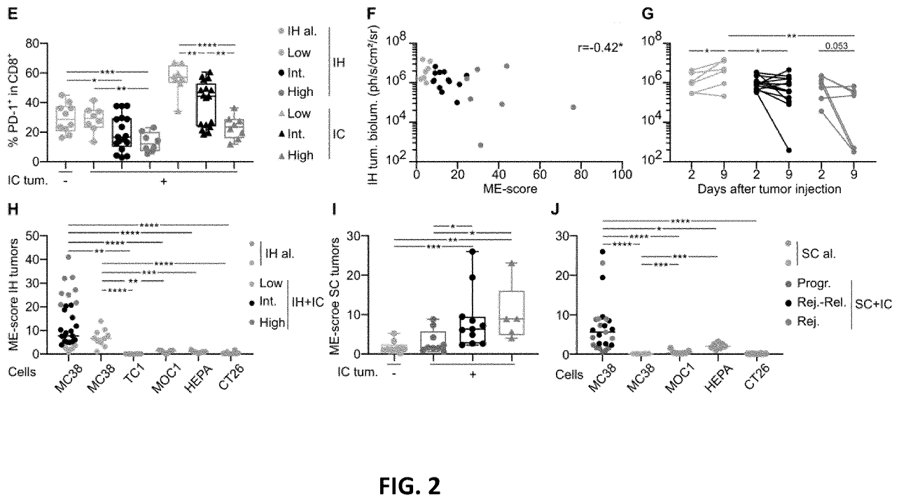

Analysis of tumor-infiltrating immune cell populations from day (D)10 samples showed increased infiltration by CD8⁺ T lymphocytes in IH tumors when co-implanted with IC tumors. Considering the central antitumor role of CD8⁺ T lymphocytes in CRC immune response (J. Galon, et al. Science. 313, 1960-1964 (2006)), the inventors segregated mice from the IH+IC group depending on the proportion of CD8⁺ T cells among tumor-infiltrating CD45⁺ cells. When impacted by IC tumors, IH tumors with high proportions of CD8⁺ T lymphocytes exhibited high proportions of DCs and low proportions of macrophages. The inventors calculated a MicroEnvironment score (ME-score) taking into account the proportions of IH tumor-infiltrating CD8⁺ T cells, DCs and macrophages (FIG. 2A). The proportions of CD8^{'} TILs and DCs were positively correlated with the ME-score whereas those of macrophages exhibited an inverse correlation with the ME-score and all correlations held true in IH tumors, where the ME-score was calculated, and in concomitant IC tumors (FIG. 2, B to D). CD8 T-cell exhaustion inversely correlated with the ME-score (FIG. 2E). In addition, high ME-score was associated with increased CD8/Treg ratio, increased proportions of conventional (Foxp3neg) CD4⁺ T cells (Tconv) and increased MHC-II expression in macrophages, as well as with decreased proportions of MDSCs and decreased CD80 expression in macrophages. At early timepoints, i.e. days 7 and 9, the ME-score was significantly inversely correlated with IH-tumor bioluminescent emission (FIG. 2F), suggesting that the ME-score could be an early predictor of progressive and rejecting CRC. Indeed, low ME-scores were significantly associated with early increase in IH tumor bioluminescence implying future progressive CRC outcome, whereas high ME-scores were related to decrease in bioluminescence, likely indicating initiation of tumor elimination (FIG. 2G). Of note, IH alone implantation of multiple syngeneic tumor cell lines led to tumor progression in all implanted mice and ensuing tumors systematically exhibited low ME-scores (FIG. 2H).

To unambiguously confirm the predictive value of the ME-score, based on the SC+IC model (FIG. 1C), the inventors implanted mice with 2 SC tumors, whereby one was surgically removed on day 10 for ME-score assessment and the second, in the contralateral flank, was monitored for tumor growth. The latter allowed for the segregation of the previously observed 3 groups of mice (i.e. progressing, rejecting-relapse and rejecting, FIG. 1C). Based on these fates, tumors removed from the same mice for immune infiltrate analysis were respectively named future progressive, future rejecting-relapse and future rejecting. Future rejecting SC tumors were highly infiltrated by CD8⁺ T cells and DCs and exhibited decreased infiltration by macrophages tallying high ME-score. Whereas PD-1 expression in CD8⁺ T cells, CD8/Treg ratio and Tconv infiltration in future rejecting SC tumors behaved similarly to high ME-score IH tumors (FIG. 2E), the inventors did not observe a full parallel in immune infiltration between IH and SC tumors impacted by distant IC tumors. All tumors from the future progressive group exhibited low ME-scores whereas high ME-scores were found in tumors from the future rejecting-relapse and future rejecting groups (FIG. 21). Finally, as for IH tumors (FIG. 2H), all SC alone tumors generated by implantation of other syngeneic cell lines, which never led to spontaneous regression (L. Tran, et al. Cancer Immunol. Res. 5, 1141-1151 (2017); C. Devaud, et al. Mol. Ther. J. Am. Soc. Gene Ther. 22, 18-27 (2014); T. Kimura, et al. Cancer Sci. 109, 3993-4002 (2018)), exhibited low ME-scores (FIG. 2J). Together, the present results uncover a colon-dependent and immune-mediated antimetastatic effect which can be predicted by the ME-score.

### 2.2 Colon-Initiated Tumor-Specific Systemic Immune Response is Associated With the Antimetastatic Effect.

Given that systemic immunity is required for effective anticancer immune surveillance (M. H. Spitzer, et al. Cell. 168, 487-502.e15 (2017)) and that the colon-governed antimetastatic effect shaped distant tumor sites, the inventors analyzed the peripheral (non-tumoral) immune response in the spleen, blood and lymph nodes (LN). In IH+IC mice with high ME-score tumors, increased proportions of CD8⁺ T cells were detected in the spleen, blood as well as in LN draining both IC and IH tumors. Although lower exhaustion levels were detected in peripheral CD8⁺ T cells, as compared to CD8⁺ TILs (FIG. 2E), immune checkpoint expression appeared increased in CD8⁺ T cells from mice with low ME-score tumors. Assessment of the proportions of total CD8⁺ T cells in mice with late stage (D27) progressive tumors, that were likely low ME-score at early stages, did not show increase in their proportions along time, neither in tumors nor in the periphery. In addition, CD8⁺ T cells maintained high exhaustion levels in late stage IH and IC tumors as in early low ME-score tumors.

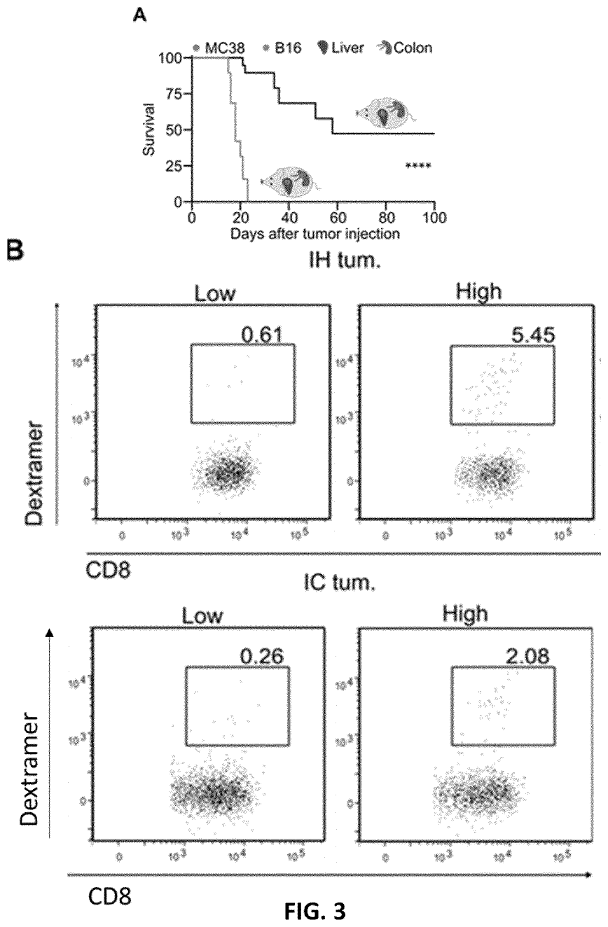

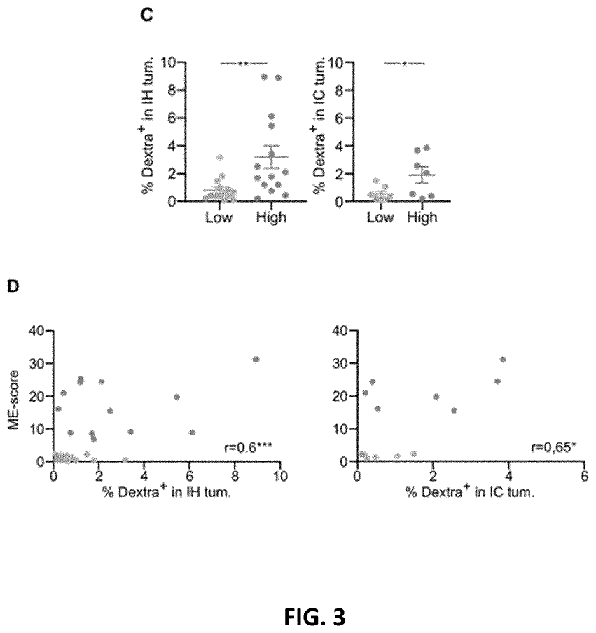

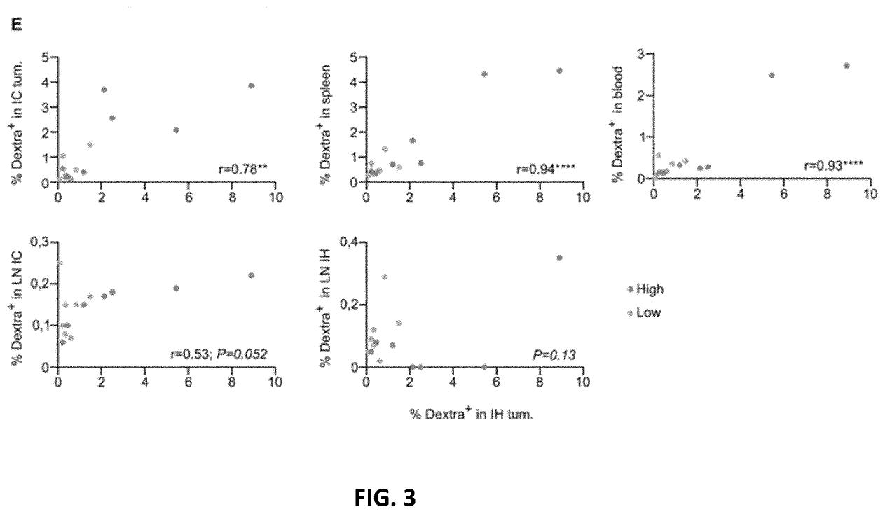

MC38 colon tumors did not trigger rejection of distant B16F10 IH tumors (FIG. 3A) suggesting that the antimetastatic effect relied on antigen specificity, possibly through colon-derived tumor-specific T cells. The inventors therefore assessed tumor-infiltrating and systemic MC38 neoantigen-specific CD8⁺ T cells (M. Yadav, et al. Nature. 515, 572-576 (2014)) using fluorescent MHC-peptide dextramers in mice bearing low and high ME-score tumors. The frequency of dextramer⁺ cells among CD8⁺ T cells was increased in high ME-score IH and IC tumors (FIG. 3, B and C) and was positively correlated with the ME-score (FIG. 3D) indicating the implication of MC38-specific CD8⁺ T cells in IH and IC tumor rejection. Tumor-specific CD8⁺ TILs from low ME-score tumors exhibited higher immune checkpoint expression suggesting inhibition of their antitumor functions and accumulated in late-stage progressive tumors without mediating tumor-rejection. When the inventors assessed the proportions of MC38-specific CD8⁺ T cells in the periphery, no significant differences were detected between IH+IC mice bearing low or high ME-score tumors. However, the inventors established positive correlations between the proportions of dextramer cells among CD8⁺ T cells infiltrating IH tumors and those detected in IC tumors as well as in peripheral locations including spleen, blood and mesenteric LN (FIG. 3E). Tumor-specific CD8⁺ T-cell proportions in IH tumors did not correlate with their proportions in hepatic LN (FIG. 3E) highlighting further the possible colon origin of CD8⁺ T cells involved in IH metastases rejection.

2.3 Colon-Antimetastatic Effect Involves Tumor-Specific α4β7⁺CD8⁺ T Lymphocytes

The inventors performed high-dimensional profiling of sorted CD45⁺ cells by scRNA-seq in order to assess the transcriptional profiles of immune populations infiltrating low, intermediate and high ME-score IH MC38-tumors as well as control IH alone tumors.

D10 IH MC38-tumors from IH+IC mice were analyzed by flow cytometry and selected in order to pool 7 high ME-score, 7 intermediate ME-score and 7 low ME-score. Seven tumors from control IH alone mice were also pooled. CD45 cells isolated from the ensuing 4 samples were subjected to scRNAseq and resulted in 2500 to 5600 analyzable cells per sample, with a coverage of approximately 20000 reads per cell.

DC in high ME-score tumors were likely conventional (c)DC1 (expressing (Clec9a) and plasmacytoid (p)DC (expressing Siglech, Ly6c2, Bst2, Tef4, Irf8 and Bel11a).

The inventors identified six clusters of macrophages (mΦ_c) and one cluster of monocytes. In control and low ME-score tumors, ˜30% of CD45⁺ tumor-infiltrating cells were mΦ_c1, reaching up to 60% when added to mΦ_c2 and mΦ_c3. mΦ_c1, mΦ_c2 and mΦ_c3 expressed the highest levels of immunosuppressive type 2 macrophage-related markers including Tgm2, Chil3 (mainly in mΦ_c1 and monocytes), Fn1, Tgfb1, Tgfbi as well as Arg1 and Mmp12 (higher in mΦ_c3). mΦ_c4 was the only cluster increased among CD45⁺ tumor-infiltrating cells in intermediate and high ME-score tumors and was negative for Cx3cr1 and expressed high levels of Cd5l, Clec4f, Vsig4, Timd4, all being markers of mouse Kupffer cells.

Among lymphoid cell clusters, NK_c1 expressed cytotoxicity-related genes (Gzma, Prf1, Eomes, Nkg7, Ncr1, Gzmb) whereas NK_c2 expressed Xcl1.Treg, which infiltrated highly control and low ME-score tumors, expressed high levels of Gzmb, Ctla4 and Il10 supporting their high suppressive abilities in these tumors.

High ME-score tumors were mainly infiltrated by CD8_c1 which expressed high levels of activation and cytotoxicity markers including Ccl5, Itgb1, Ahnak, Cx3cr1 and Gzmm.

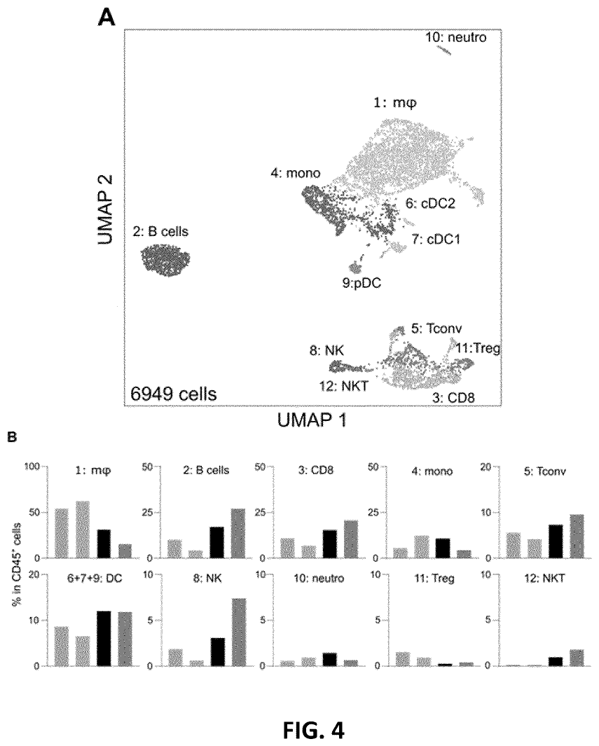

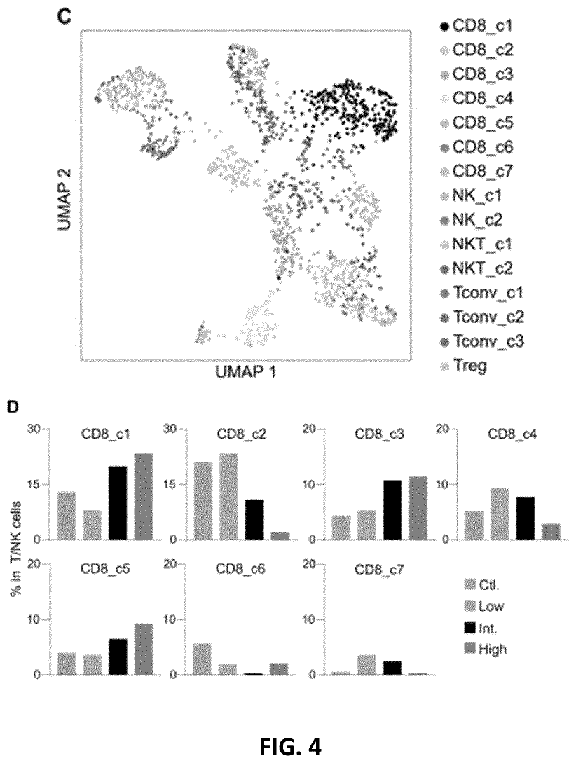

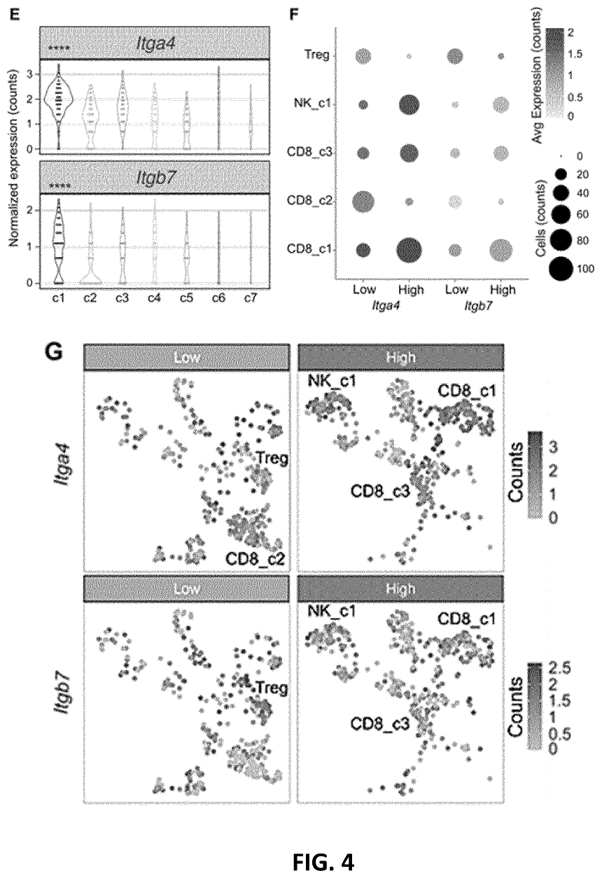

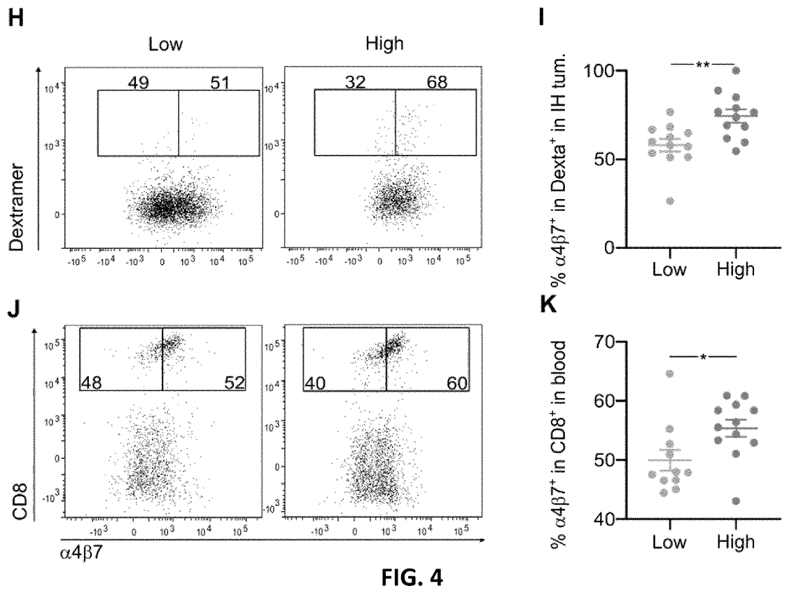

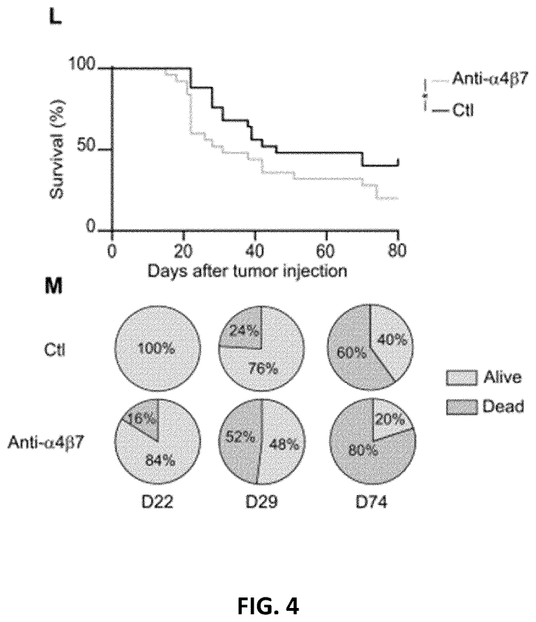

CD8_c3, also highly infiltrating high ME-score tumors, expressed high levels of genes related to CD8⁺ T-cell effector and cytotoxic functions (Gzmb, Id2, Cd226, Ccl5, Cxcr3, Tnfsf10, Ccl3) (FIG. 4D). Finally, CD8_c5 was characterized by expression of naïve T-cell markers (Sell, I17r, Tcf7, Lef1, Klf2, Ccr7, Id3) as well as typical T-cell gut homing markers including Itgae and Ccr9. On the opposite, CD8_c2 represented the major CD8⁺ T-cell subset infiltrating control and low ME-score tumors and, in addition to increased expression of cytotoxicity markers (Tnfrsf9, Prf1, Nkg7, Ifngr1, Tnfrsf18), it expressed the highest levels of exhaustion markers (Lag3, Pdcd1, Ctla4, HavCr2, Tox, Nr4a2, Nfat5, Batf, Prdm1). Low ME-score tumors were also highly infiltrated by proliferating cells expressing Mki67 (CD8_c4), which also expressed genes related to dividing cells (FIG. 4D).

The inventors identified 12 transcriptional groups corresponding to lymphoid and myeloid cell subsets, including components from the ME-score (FIGS. 4, A and B). The inventors confirmed, at the transcriptional level, the proportions of the ME-score components with a RNA ME-score that included increased CD8⁺ T-cell and DC infiltration and decreased proportions of macrophages in high ME-score tumors (FIG. 4B). Other immune cell populations varying in proportion according to the ME-score were uncovered by these analyses. Namely, antitumor B, Tconv, NK and NKT cell proportions were higher in intermediate and high ME-score tumors as compared to low ME-score and control tumors (FIG. 4B). In depth analysis of the identified cell populations showed that DCs in high ME-score tumors were enriched in conventional (c)DC1 and plasmacytoid (p)DC subsets. In addition, the inventors identified clusters of macrophages (mφ_c1, c2 and c3) that highly infiltrated low ME-score tumors and expressed the highest levels of immunosuppressive type 2 macrophage related markers. Sub-clustering of T and NK cells uncovered 7 clusters of CD8⁺ T cells with CD8_c1, CD8_c3 and CD8_c5 being increased in intermediate to high ME-score tumors (FIGS. 4, C and D). Two clusters of NK and of NKT cells were more abundant in high ME-score tumors, whereas control and low ME-score tumors were more infiltrated by Treg and Tconv as compared to high ME-score tumors. CD8_c1 and CD8_c3 expressed high levels of genes related to CD8⁺ T-cell activation and effector cytotoxic functions, including Ccl5, Itgb1, Gzmb and Id2 (A. M. van der Leun, Det al. Nat. Rev. Cancer. 20, 218-232 (2020); D. Matza, et al. Proc. Natl. Acad. Sci. U. S. A. 106, 9785-9790 (2009); B. P. Nicolet, et al. Proc. Natl. Acad. Sci. U. S. A. 117, 6686-6696 (2020)), whereas CD8_c2, the main CD8 cluster infiltrating control and low ME-score tumors, showed features of exhausted CD8⁺ T cells (Z. Chen, et al. Immunity. 51, 840-855.e5 (2019); N.

Chihara, et al. Nature. 558, 454-459 (2018)) (FIG. 4D). CD8_c1 expressed Itga4 and Itgb7, at higher levels than the other CD8 clusters (FIG. 4E).

Itga4 and Itgb7 encode α4β7 integrin, expression of which in T cells is restricted to gut-associated lymphoid tissue (GALT)-primed T cells (J. R. Mora, et al. Nature. 424, 88-93 (2003)), suggesting the colon origin of CD8_c1 cells in high ME-score IH tumors. Integrin α4β7 was described as a gut-tropic molecule as it is only acquired by T cells when activated by intestinal DCs in mesenteric LN (N. Wagner, J. Lohlert, E. J. Kunkel, K. Ley, K. Rajewsky, W. Muller, Critical role for P7 integrins in formation of the gut-associated lymphoid tissue. 382, 5 (1996); C. Berlin, et al. Cell. 74, 185-195 (1993); A. Stock, et al. Cell Biol. 91, 240-249 (2013)). It binds Mucosal Addressing Cell Adhesion Molecule-1 (MAdCAM-1), which is constitutively expressed on high endothelial venules (HEV) of GALT, including Peyer patches and mesenteric (m)LN, as well as on postcapillary venules of the gut lamina propria (C. Berlin, et al. Cell. 74, 185-195 (1993)). As compared to all lymphoid clusters, expression of Itga4 and Itgb7 genes was most prominent in cytotoxic CD8_c1 and CD8_c3 as well as NK_c1 from high ME-score tumors whereas they were mainly expressed by exhausted CD8_c2 and Treg in low ME-score tumors (FIGS. 4, F and G). The inventors extended these findings at the protein level by performing flow cytometry analyses which showed higher expression of α4β7 in tumor antigen-specific CD8⁺ T cells in high ME-score tumors (FIG. 4H and I) and revealed increased expression of α4β7 in circulating CD8⁺ T cells in the same mice (FIGS. 4J and K). In addition, whereas increased proportions of Treg and NK cells were detected in low and high ME-score tumors respectively, no differences in expression of α4β7 integrin were detected in these cells between low and high ME-score tumors, confirming scRNA-seq results (FIG. 4F). Results presented above uncovered a possible central role for α4β7 CD8⁺ T cells in the spontaneous rejection of colon tumors and the concomitant antimetastatic effect. Treatment with depleting anti-α4β7 monoclonal antibodies, which led to the reduction of the proportion of α4β7 cells in the tumor and peripheral compartments of treated mice, resulted in significantly reduced survival of IC+IH mice in comparison to non-treated control mice (FIGS. 4, L and M).

These results support the concept that colon tumors are able to generate an antitumor immune response involving specific effector α4β7 CD8⁺ T cells, which, when migrating to liver metastases, mediate metastatic tumor elimination.

### 2.4 Colon Tumors and α4β7 CD8⁺ T Cells Confer a Therapeutic Benefit for Immune Checkpoint Blockade-Mediated Control of Liver Metastases.

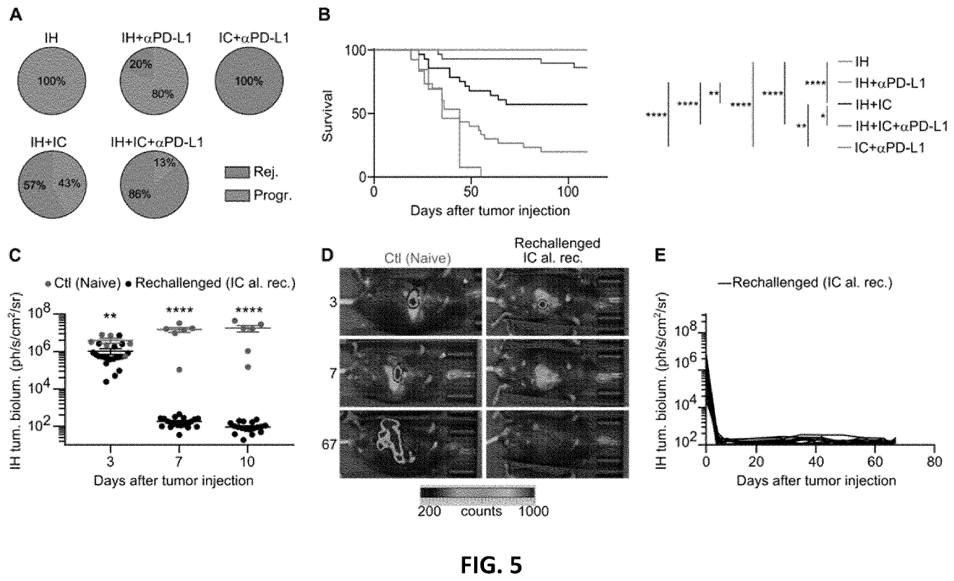

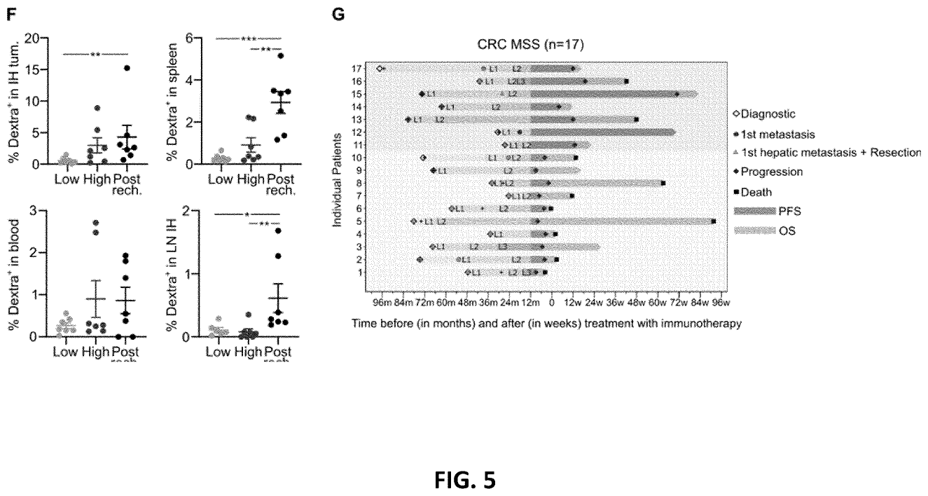

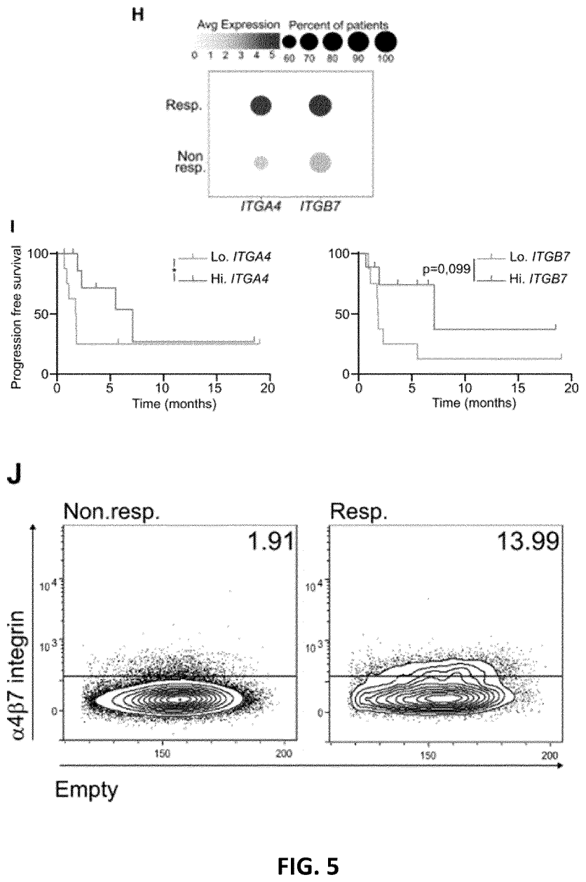

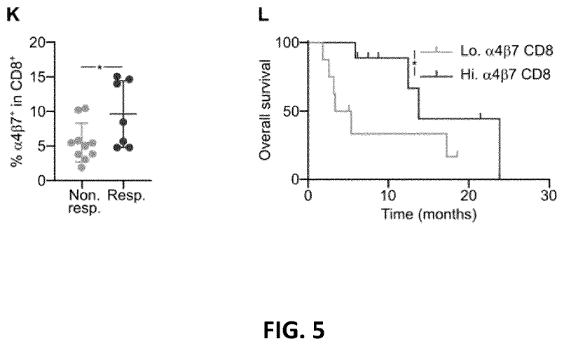

Liver metastases are particularly resistant to immunotherapy, drastically reducing ICB efficacy. Accordingly, in the present model, MC38 liver metastases systematically led to progressive lethal tumors with limited response to anti-PD-L1 proof-of-concept immunotherapy, whereas IC-located MC38 tumors were fully sensitive to anti-PD-L1 treatment (FIGS. 5, A and B). The presence of a distant IC primary tumor triggered IH metastases rejection more effectively than immunotherapy as confirmed by prolonged IH+IC mice survival (FIGS. 5, A and B) and reduced tumor size as compared to IH mice treated with anti-PD-L1. The highest efficacy of immunotherapy was observed when IH tumor-bearing mice had a concomitant IC tumor during treatment (FIGS. 5, A and B), suggesting that IC tumors play a beneficial role in IH tumors for response to immunotherapy. Efficacy of immunotherapy by ICB relies on preexisting antitumor effector and memory responses (A. D. Waldman, et al. Nat. Rev. Immunol., 1-18 (2020)). To investigate the memory immune response generated by IC tumors, the inventors rechallenged IH+IC mice from the tumor rejecting group, which remained tumor free for at least 41 days, by implanting MC38 cells IH. Despite initial tumor take in all mice, 100% of them rapidly and fully rejected their IH tumors, supporting the persistence of a long-lived memory response following initial rejection. Rejection of IC tumors implanted without concomitant IH tumors was sufficient to protect mice from IH tumor rechallenge (FIG. 5, C to E). In these rechallenged rejecting mice, increased proportions of MC38 neoantigen-specific CD8⁺ T cells were detected in the liver, at the scar of the rejected IH tumor, as well as in the spleen, the blood and hepatic LNs as compared to the same compartments analyzed D10 in IH+IC mice bearing low and high ME-score tumors (FIG. 5F), suggesting their involvement in protecting the liver from tumor challenge.

To investigate the relevance of the ME-score and of α4β7 CD8⁺ T cells in patient response to immunotherapy, the inventors analyzed, by RNA-seq, metastatic lesions from MSS mCRC patients treated by ICB in combination with targeted agents (FIG. 5G). Seven out of 17 patients in the cohort experienced clinical benefit (FIG. 5G). In metastatic lesions, levels of ITGB7 and ITGA4 expression, encoding human α4β7 integrin, positively correlated with expression of genes related to CD8⁺ T cell cytotoxicity and exhaustion, which levels of expression were previously described as being increased in primary lesions responding to immunotherapy (T. N. Gide, et al. Cancer Cell. 35, 238-255.e6 (2019); C.-C.

Balança, et al. Cancer Immunol. Res. 8, 869-882 (2020)). Indeed, the proportion of patients whose metastatic tumors expressed ITGB7 and ITGA4 was higher among responders than non-responders and expression levels of both genes were higher in responding metastases (FIG. 5H). In addition, progression free survival (PFS) was increased in patients expressing higher levels of ITGA4 in their metastases and the same trend was found for ITGB7 (FIG. 5I), suggesting a beneficial role of α4β7 integrin expressing CD8⁺ T cells in patient metastases response to immunotherapy. Finally, the inventors found increased proportions of cells expressing α4β7 among pretreatment circulating CD8⁺ T cells from responder as compared to non-responder patients (FIGS. 5, J and K). Increased proportions of circulating α4β7 CD8⁺ T cells were associated with increased overall survival (OS) (FIG. 5L). Data obtained in the clinical cohort support conclusions drawn from the preclinical model regarding the involvement of colon-derived effector α4β7⁺CD8⁺ T cells in metastases response to immunotherapy and in patient outcome.

Altogether these results show that the colon-initiated immune response, involving α4β7⁺ CD8⁺ T cells confers systemic protection in the immunotherapy settings.

### Discussion

The present work brings to light the potential of mucosal immunity to intestinal tumors to exert systemic functions and to shape extra-intestinal tumors microenvironment and outcome. The two-tumor model, in which liver metastases regressed consequently to a colon-dependent immune-mediated process, beds this concept. The colon antimetastatic effect is further supported by the colon memory immune response which protected liver from tumor challenge and by the ability of colon tumors to increase liver metastases response to immunotherapy. The ME-score, which reflected the antimetastatic effect, was delineated, in liver metastases, by both anti-and protumor immune cell populations. Within the CD8⁺ T-cell population, which was one of the ME-score components, enterotropic cells expressing α4β7 integrin were key players of the colon antimetastatic effect. Enterotropism of this lymphocyte subset was previously established, explained by a gut vasculature-specific expression of the α4β7 integrin ligand, MAdCAM-1 (N. Wagner, J. Lohlert, E. J. Kunkel, K. Ley, K. Rajewsky, W. Muller, Critical role for P7 integrins in formation of the gut-associated lymphoid tissue. 382, 5 (1996); C. Berlin, et al. Cell. 74, 185-195 (1993); M. Briskin, et al. Am. J. Pathol. 151, 97-110 (1997)). Considering their gut localization, α4β7⁺CD8⁺ T cells have been extensively studied for their role in driving pathological inflammation in inflammatory bowel disease (IBD) (P. E. Hesterberg, et al. Gastroenterology. 111, 1373-1380 (1996); D. K. Podolsky. N. Engl. J. Med. 325, 1008-1016 (1991); D. K. Podolsky, et al. J. Clin. Invest. 92, 372-380 (1993)). Their implication in gastrointestinal cancers remains virtually unexplored. Nonetheless, two recent studies described the implication of β7 integrin-expressing cells in promoting inflammatory CRC tumor initiation (S. Das, et al. PloS One. 13, e0204181 (2018)) and in the control of established primary CRC tumor growth (Y. Zhang, R. et al. Cancer Immunol. Res. 9, 967-980 (2021)), without exploring the potential role of gut-derived α4β7⁺ CD8⁺ T lymphocytes in cancer immunosurveillance. In line with the present work, pinpointing α4β7⁺CD8⁺ T lymphocytes relocation to the liver, IBD-associated extra-intestinal disorders, which include collateral liver inflammation, were linked to a destructive influx of inflammatory α4β7⁺ CD8+ T cells (D. H. Adams, et al. Nat. Rev. Immunol. 6, 244-251 (2006); B. Eksteen, et al. J. Exp. Med. 200, 1511-1517 (2004)). Although mechanisms explaining relocation of enterotropic α4β7⁺CD8+ T cells to the liver are still unclear, a pathological upregulation of MAdCAM-1 in hepatic vasculature endothelial cells was evoked (D. H. Adams, et al. Nat. Rev. Immunol. 6, 244-251 (2006)). The present work demonstrates that colon-primed tumor-specific α4β7⁺CD8+ T cells have the potential to relocate to an extra-intestinal tumor, highlighting a central role for gut local immunity in cancer immunosurveillance. The intestine contains the largest number of immune cells in the body and was long considered as hosting gut-restricted immune processes (S. Shalapour, et al. Annu. Rev. Immunol. 38, 649-671 (2020)). However, there is increasing evidence of intestinal influence on physiological and pathological processes throughout the body, dependent, for instance, upon the gut microbiome or dietary constituents (A. Albillos et al. J. Hepatol. 72, 558-577 (2020); D. H. Adams, et al. Nat. Rev. Immunol. 6, 244-251 (2006); B. Eksteen, A et al. J. Exp. Med. 200, 1511-1517 (2004)). The present results open a new perspective on the extra-intestinal impact of the gut, through mucosal immune cell populations participating in systemic cancer immunosurveillance.

In the preclinical setting, spontaneous rejection of implanted tumors was not observed in any other location than the colon (G. Trimaglio, et al. Oncoimmunology. 9, 1790125 (2020), C. Devaud, et al. Mol. Ther. J. Am. Soc. Gene Ther. 22, 18-27 (2014)), emphasizing the central role of enterotropic α4β7⁺CD8⁺ T cells in this phenomenon and, a fortiori, in the ensuing antimetastatic effect. α4β7⁺CD8⁺ T cells in metastases and in the circulation may therefore represent biomarkers of the colon-derived systemic antimetastatic effect. In the present model, α4β7⁺CD8⁺ T cells relocating from concomitant colon tumors to liver metastases increased the response of the latter to ICB, supporting the investigation of α4β7⁺CD8⁺ T cells as predictive biomarkers of response to immunotherapy in cancer patients. In line with this postulate, high expression of ITGB7 gene in tumors was associated with response to therapy and survival in lung and urothelial cancer patients receiving ICB (Y. Zhang, et al. Cancer Immunol. Res. 9, 967-980 (2021)). While ICB showed dismal efficacy for mCRC patients treatment (K. Ganesh, et al. Nat. Rev. Gastroenterol. Hepatol. 16, 361-375 (2019)), its use in the neoadjuvant setting at early CRC stages was associated to pathological responses (M. Chalabi, et al. Nat. Med. 26, 566-576 (2020)). The assessment of α4β7⁺CD8⁺ T cells in this setting would ascertain their role in tumor rejection and interest as biomarkers of response. It would also lay the ground for neoadjuvant immunotherapy of advanced stage CRC patients bearing α4β7⁺CD8⁺ T cells rich colon tumors, the antimetastatic potential of which would be enhanced by ICB. Finally, elicitation of tumor-specific enterotropic α4β7⁺CD8⁺ T cells through vaccine strategies (S. I. Hammerschmidt, et al. J. Clin. Invest. 121, 3051-3061 (2011)) could lead, based on the present model, to enhanced tumor rejection and sensitivity to ICB, in both the primary colon and metastatic locations. The relevance of such strategies could be extended beyond CRC in view of the favorable association between response to ICB and ITGB7 gene expression in other tumor types (Y. Zhang, et al. Cancer Immunol. Res. 9, 967-980 (2021)).

1. A method for determining the response of a metastasis to immunotherapy in a metastatic colorectal cancer patient, said method comprising:
i) determining the density of CD8⁺ T cells, dendritic cells and macrophages in a patient sample,
ii) calculating a MicroEnvironment score (ME-score) corresponding to the ratio of the density of CD8⁺ T cells and dendritic cells to the density of macrophages,
wherein a higher value of the ME-score in comparison to a control value is indicative that the metastasis of the patient is responsive to immunotherapy.

2. The method of claim 1 further comprising:
i) determining the density of B-cells, NK cells and/or regulatory T cells in a patient sample,
ii) calculating a MicroEnvironment score (ME-score) based on the ratio of density of CD8⁺ T cells, dendritic cells, B cells and/or NK cells on the density of macrophages and preferably regulatory T cells.

3. The method of claim 1 wherein said CD8⁺ T cells are enterotropic CD8⁺ T cells, preferably α4β7⁺CD8⁺T cells.

4. A method for determining the response of a metastasis to immunotherapy in a metastatic colorectal cancer patient, said method comprising determining the density of colon-derived T cells, preferably α4β7⁺ CD8⁺ T cells in a patient sample, wherein a higher density of said colon-derived T cells, preferably α4β7⁺ CD8⁺ T cells in comparison to a control value is indicative that the metastasis of the patient is responsive to immunotherapy.

5. The method according to claim 1 wherein said dendritic cells are CD11c⁺ cells.

6. The method according to claim 1 wherein said macrophages are CD68⁺ cells

7. The method according to claim 1 wherein the density of cells is determined by flow cytometry, immunofluorescence, immunohistochemistry, single cell RNA seq, bulk RNA-Seq and qRT-PCR.

8. The method according to claim 1 wherein said patient sample is tumor tissue or biological fluid sample, preferably metastatic tumor tissue or patient blood sample such as patient peripheral blood mononuclear cells.

9. An immunotherapy for use in the treatment of a metastatic colorectal cancer in a patient in need thereof wherein said immunotherapy is administered in said patient previously identified as having metastasis responsive to immunotherapy in a method according to claim 1.

10. A pharmaceutical composition comprising an immune-stimulatory agent for use in the treatment of a metastatic colorectal cancer in a patient in need thereof wherein said immune-stimulatory agent is administered in said patient previously identified as having metastasis non-responsive to immunotherapy in a method according to claim 1.

11. The pharmaceutical composition for use of claim 10 wherein said immune-stimulatory agent is a colorectal cancer vaccine, preferably able to elicit tumor-antigen specific α4β7⁺CD8⁺ T cells.

12. The pharmaceutical composition for use of claim 10 further comprising retinoic acid.

13. A pharmaceutical composition comprising an immune-stimulatory agent, preferably a colorectal cancer vaccine and retinoic acid for use in the treatment of a metastatic colorectal cancer in a patient in need thereof.

14. A kit for determining the response of a metastasis to immunotherapy in a metastatic colorectal cancer patient comprising anti-α4β7 and anti-CD8 antibodies, preferably anti-α4β7, anti-CD8, anti-CD11c and/or anti-CD68 antibodies.

15. A kit for determining the response of a metastasis to immunotherapy in a metastatic colorectal cancer patient comprising a pair of primers for amplifying at least one gene marker of CD8⁺ T cells, preferably at least one gene marker of CD8⁺ T cells and at least one gene marker of α4β7⁺ cells, more preferably at least one gene marker of CD8⁺ T, at least one gene marker of dendritic cell and at least one gene marker of macrophages, again more preferably at least one gene marker of CD8⁺ T cells and at least one gene marker of α4β7+ cells, at least one gene marker of dendritic cell and at least one gene marker of macrophages.
